(19) United States
(12) Patent Application Publication
(10) Pub. No.: US 2017/0240901 A1
(43) Pub. Date: Aug. 24, 2017

(54) NUCLEIC ACIDS, AND USES THEROF

(71) Applicant: The University of Liverpool, Liverpool (GB)

(72) Inventors: John Hunt, Liverpool, Merseyside (GB); John Innes, Liverpool, Merseyside (GB)

(73) Assignee: The University of Liverpool, Liverpool (GB)

(21) Appl. No.: 15/121,742

(22) PCT Filed: Feb. 26, 2015
(86) PCT No.: PCT/GB2015/050552

§ 371 Date: Aug. 25, 2016

(30) Foreign Application Priority Data
Feb. 26, 2014 (GB) 1403359.1
Feb. 26, 2014 (GB) 1403360.9

(51) Int. Cl. C12N 15/113; A61K 31/713; A61K 9/50

(52) U.S. Cl. CPC C12N 15/1136; A61K 9/5031; A61K 31/713; C12N 2310/14; C12N 2320/32

(57) ABSTRACT

Provided are siRNA molecules that are particularly efficient in their ability to reduce transcription and expression of TNF or IL-1β in canine macrophage and synovial cells. The siRNA molecules demonstrate improved silencing of TNF or IL-1β as compared to siRNA molecules disclosed prior to the present invention. These properties are demonstrated in accepted experimental models of osteoarthritis, and particularly canine osteoarthritis. The siRNA molecules of the invention, may be employed in pharmaceutical compositions for use in the prevention and/or reduction of osteoarthritis in dogs. The inventors have found that encapsulation of the siRNAs in small PLGA microspheres confers surprisingly advantageous properties.

The present invention relates to siRNA molecules that silence tumour necrosis factor and to the medical use of such siRNA molecules in the prevention and/or treatment of canine osteoarthritis. The present invention also relates to siRNA molecules that silence interleukin-1β, and to the medical use of such siRNA molecules in the prevention and/or treatment of canine osteoarthritis. Further, the present invention also relates to the combination of siRNA molecules that silence tumour necrosis factor and siRNA molecules that silence interleukin-1β, and to the medical use of such a combination in the prevention and/or treatment of canine osteoarthritis. The invention also relates to pharmaceutical compositions comprising such siRNA molecules, and to methods of preventing and/or treating canine osteoarthritis utilising the siRNA molecules or pharmaceutical compositions of the invention.

### INTRODUCTION

Osteoarthritis is the most common form of arthritis in dogs, and is particularly prevalent in aged animals. It is a localised disease that results in the degeneration and destruction of articular cartilage in joints such as the shoulder, elbow, knee, or hip. Recent data indicate that synovitis is crucial for both the structural and symptomatic progression of the disease. There is robust and extensive evidence that catabolic cytokines, such as tumour necrosis factor and interleukin-1β, produced by macrophages in the synovial lining layer, are central to the pain, inflammation and progression of osteoarthritis.

Tumour necrosis factor (also referred to as TNF or TNF-α) is an important inflammatory cytokine implicated in a range of inflammatory conditions including neoplasia, inflammatory bowel disease, and arthritis. Arthritis, in its various forms, involves an inflammatory response from synovium and a catabolic response in articular cartilage. TNF has been heavily implicated in both processes in both canine immune-mediated polyarthritis and canine osteoarthritis.

TNF is produced predominantly by activated macrophages and the primary role of TNF is in the regulation of the acute phase response and regulation of immune cells. TNF can bind two cell-surface receptors, TNF-R1 and TNF-R2. TNF-R1 is expressed in most tissues, and can be fully activated by both the membrane-bound and soluble trimeric forms of TNF, whereas TNF-R2 is found only in cells of the immune system, and respond to the membrane-bound form of the TNF homotrimer. Binding of TNF to cell-surface receptor can induce activation of NF-κB, activation of the MAPK pathways, and cell-death signalling.

Interleukin-1β (IL-1β) is a member of the interleukin 1 cytokine family, activity of which is associated with the body's immune response to insults. It is also involved in a several cellular responses, including cell proliferation, differentiation, and apoptosis. Its expression is induced by stimulation of inflammatory cells, such as macrophages or dendritic cells, for example by lipopolysaccharide.

In all forms of arthritis, the synovium proliferates and is infiltrated with inflammatory cells, such as macrophages. In osteoarthritis, there is an increasing interest in the role of synovitis (Bondeson et al., 2010), not only with respect to the pain associated with the disorder (Baker et al., 2010), but also with respect to disease progression as measured by loss of articular cartilage matrix (Ayral et al., 2001; Roemer et al., 2011) through catabolic proteolytic activity by degradative enzymes such as matrix metalloproteinases (e.g. MMP-13) and ‘aggrecanases’ (e.g. ADAMTS-4, -5). Inflammatory cytokines from synovial cells may initiate an inflammatory cascade and may also act in a paracrine fashion to stimulate catabolic activity in articular chondrocytes. However, direct production of degradative enzymes by synovial cells which may act on articular cartilage is another possible way in which synovium may contribute to disease progression.

RNA interference (RNAi) (Fire et al., 1998) involves interruption of mRNA translation and can be caused by small interfering RNA (siRNA) (Hamilton and Baulcombe, 1999). Synthetic siRNAs were shown to be able to induce RNAi in cultured mammalian cells (Elbashir et al., 2001) and the therapeutic effects of siRNA in mice with experimentally-induced arthritis have been reported (Khoury et al., 2008; Schiffelers et al., 2005). siRNAs are double stranded RNA fragments which, when introduced in to the cell, combine with other cellular factors to produce an RNA-induced silencing complex (RISC) which unwinds the double stranded RNA. One strand guides the RISC to cleave the target mRNA in a sequence-specific manner leading to translational repression of the target gene. Whilst siRNA molecules are widely available for suppression of many genes in human and rodent cells, such tools are not generally available for canine cellular studies.

SiRNA-mediated gene silencing offers the ability to silence individual gene expression with a high degree of specificity. SiRNA is already being widely used in basic science as a method to study the functions of genes and it may provide an alternative therapeutic promise to novel therapies in the future.

Although siRNAs hold great potential as a new treatment modality due to efficient and specific gene silencing, significant challenges remain. The main challenges are the methods to deliver siRNAs to the target cells and to extend the duration of the therapeutic effect. Therefore, the development of a carrier for siRNA delivery not only for targeted delivery but also to protect siRNA from degradation by nuclease is important before siRNA can achieve widespread clinical use.

SiRNA can be administrated by systemic delivery strategies or local delivery strategies; local delivery strategies seem much more feasible for clinical application in the short term future. In local delivery strategies siRNA is targeted directly into a compartment (such as eye, joint cavity) to protect the siRNA from nucleases and the immune system. In the eye and articulating joints for example because of the relatively small volume in these compartments, siRNA can be delivered at a very high concentration. Therefore, most ongoing research focuses on local rather than systemic administration.

Macrophages involved in synovitis represent an ideal target cell for systems for the local delivery of siRNA due to their inherent phagocytotic capability and the cells' central role in production of inflammatory cytokines in osteoarthritis. On the other hand, recent pre-clinical and clinical studies indicate that providing a carrier of siRNA is one of the most important problems to be addressed. Various carrier systems have been investigated extensively to deliver drugs and proteins to macrophages. The most studied carriers systems are virus-based delivery systems, liposome-based and polymer-based. In spite of their higher efficiency, virus-based systems may be fatally flawed due to the safety concerns they raise as they induce mutations and trigger immunogenic and inflammatory responses. Liposome-based carriers are more attractive due to their good biocompatibility and facile manufacture. However, liposome yields relatively low transfection efficiency and there are stability problems in transport and storage.

### SUMMARY OF THE INVENTION

In a first aspect of the invention there is provided an siRNA molecule comprising a nucleic acid sequence selected from the group consisting of: SEQ ID NO. 1, SEQ ID NO. 2, SEQ ID NO. 3, SEQ ID NO. 7, SEQ ID NO. 8, SEQ ID NO. 9, and a nucleic acid sequence sharing at least 90% identity with SEQ ID NO. 1, SEQ ID NO. 2, SEQ ID NO. 3, SEQ ID NO. 7, SEQ ID NO. 8, SEQ ID NO. 9

In a second aspect of the invention there is provided an siRNA molecule in accordance with the first aspect of the invention, for use in the prevention and/or treatment of osteoarthritis in a dog. The siRNA molecule may be in accordance with any of the embodiments of the first aspect of the invention disclosed herein.

An siRNA molecule in accordance with the invention may be an isolated nucleic acid molecule.

In a third aspect the invention provides a pharmaceutical composition comprising an siRNA molecule in accordance with the first aspect of the invention and a pharmaceutically acceptable carrier. The siRNA molecule may be in accordance with any of the embodiments of the first aspect of the invention disclosed herein. The pharmaceutically acceptable carrier may advantageously comprise PLGA microspheres, as considered further below.

In a fourth aspect of the invention there is provided a method of preventing and/or treating osteoarthritis in a dog, the method comprising providing to a dog in need of such prevention and/or treatment a therapeutically effective amount of an siRNA molecule in accordance with the first aspect of the invention. The siRNA molecule may advantageously be provided in the form of a pharmaceutical composition of the invention.

### DETAILED DESCRIPTION OF THE INVENTION

The present invention is based upon the inventors' identification of siRNA molecules that are particularly efficient in their ability to reduce transcription and expression of TNF or IL-1β in canine macrophage and synovial cells. The siRNA molecules demonstrate improved silencing of TNF or IL-1β as compared to siRNA molecules disclosed prior to the present invention. These properties are demonstrated in accepted experimental models of osteoarthritis, and particularly canine osteoarthritis, and make the siRNA molecules of the invention, and pharmaceutical compositions of the invention incorporating these siRNA molecules, promising agents for use in the prevention and/or reduction of osteoarthritis in dogs.

Certain terms used throughout the specification will now be further defined in the following paragraphs.

siRNA Molecules

An siRNA molecule in accordance with the invention comprises the nucleic acid sequence of SEQ ID NO. 1, SEQ ID NO. 2, SEQ ID NO. 3, SEQ ID NO. 7, SEQ ID NO. 8, or SEQ ID NO. 9, or a nucleic acid sequence sharing at least 90% identity with SEQ ID NO. 1, SEQ ID NO. 2, SEQ ID NO. 3, SEQ ID NO. 7, SEQ ID NO. 8, or SEQ ID NO. 9. In suitable embodiments the siRNA may comprise a nucleic acid sequence sharing at least 91%, 92%, 93%, 94%, 95%, 96%, 97%, 98%, 99% identity with any one of the sequence set out in SEQ ID NO. 1, SEQ ID NO. 2, SEQ ID NO. 3, SEQ ID NO. 7, SEQ ID NO. 8 or SEQ ID NO. 9.

In a suitable embodiment an siRNA molecule in accordance with the invention may comprise a nucleic acid sequence that differs from SEQ ID NO. 1, SEQ ID NO. 2, SEQ ID NO. 3, SEQ ID NO. 7, SEQ ID NO. 8 or SEQ ID NO. 9 by a maximum of two nucleotides. Suitably an siRNA molecule of the invention may comprise a nucleic acid sequence that differs from SEQ ID NO. 1, SEQ ID NO. 2, or SEQ ID NO. 3, SEQ ID NO. 7, SEQ ID NO. 8 or SEQ ID NO. 9 by a single nucleotide.

The siRNA molecules in accordance with the invention, comprising or consisting of SEQ ID NO. 1, SEQ ID NO. 2 or SEQ ID NO. 3, or variations thereof will be effective in silencing TNF. The siRNA molecules comprising or consisting of SEQ ID NO. 7, SEQ ID NO. 8 or SEQ ID NO. 9, or variations thereof will be effective in silencing IL-1β.

As described elsewhere in the specification, siRNA molecules comprising the nucleic acid sequence of SEQ ID NO. 2 have proved to be particularly effective in silencing TNF, and so these siRNA molecules, or siRNA molecules based on this sequence (e.g. sharing at least 90 or 95% identity with SEQ ID NO. 2, or varying from SEQ ID NO. 2 by two nucleotides or a single nucleotide) represent favoured embodiments of the first aspect of the invention, and siRNA molecules particularly suited for use in the various other aspects of the invention.

Additionally, and as also described elsewhere in the specification, siRNA molecules comprising the nucleic acid sequence of SEQ ID NO. 7 have proved to be particularly effective in silencing IL-1β, and so these siRNA molecules, or siRNA molecules based on this sequence (e.g. sharing at least 90 or 95% identity with SEQ ID NO. 7, or varying from SEQ ID NO. 7 by two nucleotides or a single nucleotide) represent another favoured embodiments of the first aspect of the invention, and siRNA molecules particularly suited for use in the various other aspects of the invention.

As shown in Tables 4 and 5, SEQ ID NOs. 1, 2, 3, 7, 8 or 9 are optionally provided in the form of double-stranded siRNA molecules, in which SEQ ID NO. 1 is paired with SEQ ID NO. 4, SEQ ID NO. 2 is paired with SEQ ID NO. 5, SEQ ID NO. 3 is paired with SEQ ID NO. 6, SEQ ID NO. 7 is paired with SEQ ID NO. 10, SEQ ID NO. 8 is paired with SEQ ID NO. 11 and SEQ ID NO. 9 is paired with SEQ ID NO. 12. Unless the context requires otherwise, references in the present specification to an siRNA of the invention may be taken as encompassing the stated siRNA in single-stranded or double-stranded form. Suitable double-stranded forms of the siRNAs of SEQ ID NOs. 1, 2 or 3 may comprise the siRNA pairs shown in Table 4 and suitable double-stranded forms of the siRNAs of SEQ ID NOs. 7, 8 or 9 may comprise the siRNA pairs shown in Table 5.

It will also be appreciated that the siRNA molecules of the invention may be provided in the form of precursors, such as short hairpin RNAs, that undergo processing to yield siRNA molecules of SEQ ID NO. 1, SEQ ID NO. 2, SEQ ID NO. 3, SEQ ID NO. 7, SEQ ID NO. 8 or SEQ ID NO. 9. Such precursors are also encompassed by the references to siRNA molecules of the present invention.

Medical Uses

Another important aspect of the present invention is the medical use of the siRNA molecules disclosed herein. While the siRNA molecules of the invention are novel and inventive in themselves, additional valuable subject matter is found in the medical uses of these siRNA molecules. In particular, the siRNA molecules of the invention lend themselves to medical use in the prevention and/or treatment of osteoarthritis in dogs. These medical uses may suitably be implemented using pharmaceutical compositions of the invention, more details of which are provided elsewhere in this disclosure.

The medical uses described herein will utilise therapeutically effective amounts of the siRNA molecules of the invention. Suitable therapeutically effective amounts may be selected with reference to the desired medical use, such as the prevention of osteoarthritis in dogs, or the treatment of osteoarthritis in dogs. Therapeutically effective amounts able to effect such prevention and/or treatment may be determined by means that will be well known to those skilled in the art.

Factors that may be considered in the determination of a therapeutically effective amount of a polypeptide, variant, or nucleic acid of the invention may include: the nature of the agent in question (i.e. whether the agent is an siRNA molecule for use as a single or combination therapy, or whether the siRNA agent is provided in an encapsulated form); the activity of the agent in question; the severity of the osteoarthritis to be prevented and/or treated; the size of the subject requiring prevention and/or treatment; and the route by which the agent is to be administered.

Merely by way of example, a therapeutically effective amount of an siRNA molecule of the invention, may be between 1.5 g and 1 μg. A suitable therapeutically effective amount may be between 1500 mg and 1 mg; for example between 1000 mg and 50 mg; such as between 500 mg and 100 mg. Alternatively a suitable therapeutically effective amount may be between 100 mg and 1 mg; for example between 50 mg and 5 mg; such as between 25 mg and 10 mg. In a further suitable embodiment, a suitable therapeutically effective amount may be between 500 μg and 1 μg; for example between 400 μg and 5 μg; such as between 250 μg and 10 μg. Merely by way of example, a suitable therapeutically effective amount may be between 200 μg and 15 μg, such as between 150 μg and 20 μg, between 100 μg and 25 μg, or between 50 μg and 30 μg. Suitably a therapeutically effective amount may be approximately 40 μg.

Within a course of treatment to prevent and/or treat canine osteoarthritis, an siRNA of the invention may be provided in one or more administrations. Incidences of administration may be provided once per 24 hours, once a week, once, a month, or as otherwise required.

It will be appreciated that the preventative use of the siRNA molecules of the invention to prevent diseases such as osteoarthritis in dogs will involve the provision of siRNA molecules of the invention to a dog prior to the onset of osteoarthritis. Such prophylactic use may particularly suitable for dogs considered to be at elevated risk of developing osteoarthritis. Merely by way of example, factors that may be considered to suggest such an elevated risk of developing osteoarthritis may include one or more selected from the group consisting of: age of the subject; weight of the subject; presence of predisposing conditions (such as osteochondrosis; hip or elbow dysplasia; diabetes; or Cushing's disease); and active lifestyle of the subject (working dogs may be considered to be at an elevated risk of developing osteoarthritis).

The use of siRNA molecules of the invention to treat diseases such as osteoarthritis in dogs may involve the provision of siRNA molecules to a dog that has been diagnosed as having a disease requiring such treatment. Thus, in the case of canine osteoarthritis, siRNA molecules of the invention may be provided to a dog that has been diagnosed as having osteoarthritis. Such a diagnosis may, for example, utilise techniques such as X-rays, CT scans or MRI of an affected joint, and additionally, or alternatively, arthrocentesis or assessment of range of joint movement.

It will also be appreciated that the use of siRNA molecules of the invention to treat canine osteoarthritis may involve the provision of the siRNA molecules to a dog that exhibits symptoms consistent with osteoarthritis, even when no formal diagnosis of osteoarthritis has taken place. Merely by way of example, the siRNA molecules of the invention may be of medical use to dogs exhibiting one or more symptoms selected from the group consisting of: lethargy; reduced mobility; and pain in response to joint movement.

A number of other important and useful considerations may be taken into consideration when implementing the medical uses of the invention, and many of these considerations will also be applicable to pharmaceutical compositions of the invention (described in more detail below), which may be used to implement these medical uses.

For example, medical uses of the siRNA molecules of the invention may make use of systemic delivery strategies for the provision of the siRNA molecules to a site where they are to exert their therapeutic effect.

Alternatively, the medical uses of the invention may make use of localised delivery strategies for the provision of the siRNA molecules of the invention. It is known that macrophages of the synovium play an important role in the orchestration and maintenance of synovitis, the inflammatory of the synovium that contributes to osteoarthritis. Accordingly, localised administrations that allow the siRNA molecules of the invention to be provided to macrophages associated with synovitis in this manner represent advantageous embodiments of the medical uses of the invention.

Suitable localised delivery strategies include injection into joints requiring prevention and/or treatment of osteoarthritis, and so siRNA molecules for use in this embodiment may be provided in a form that is suited to localised injection. Medical uses of the siRNA molecules of the invention in this manner are particularly beneficial since the compartment (such as a joint) into which the localised injection is made may be relatively small, allowing very high concentrations of the siRNA molecules to be established at the site where they are required.

Alternatively, siRNA molecules of the invention may be provided via viral vectors. In a suitable example, such vectors may be able to bring about the expression of the siRNA molecules through cells at the site to which they are administered.

Medical uses of the siRNA molecules of the invention include the use of such siRNA molecules in monotherapy. In such applications the siRNA of the invention is substantially the only therapeutic agent providing for the prevention and/or treatment of canine osteoarthritis. Alternatively, the medical uses of the siRNA molecules of the invention may be uses in combination therapy, where the siRNA molecules of the invention are provided (and optionally formulated) with one or more further therapeutic agents for use in the prevention and/or treatment of canine osteoarthritis.

Suitable examples of the use of the siRNA molecules of the invention in monotherapy and in combination therapies are discussed elsewhere in the specification, particularly in the context of pharmaceutical compositions and methods of treatment using the siRNA molecules of the invention. Except for where the context requires otherwise embodiments set out in these discussions will also represent suitable embodiments that may be used in connection with the medical uses of the invention.

Pharmaceutical Compositions

Pharmaceutical compositions of the invention represent preferred means by which siRNA molecules of the invention may be provided to a subject requiring their therapeutic activity. Suitable pharmaceutical compositions of the invention may provide benefits such as stabilisation of the siRNA molecules (thus reducing degradation of the siRNA molecules prior to their use); targeted delivery of the siRNA molecules; capacity to carry large amounts of siRNA (thus allowing different therapeutic siRNA molecules to be combined in the pharmaceutical compositions); or sustained release of siRNA molecules (thus extending the duration of the therapeutic effect of the siRNA molecules at their site of action).

The pharmaceutical compositions of the invention comprise the siRNA molecules of the invention with a suitable pharmaceutically acceptable carrier. Suitable examples of pharmaceutically acceptable carriers that may be used in the pharmaceutical compositions of the invention will be apparent to those skilled in the art, and include liposome-based or polymer-based carriers. Polymers that may be used include albumin, gelatine, chitosan, dextran, polylactic acid (PLA), and polylactic-co-glycolic acid (PLGA). However, the inventors have devised specific pharmaceutical compositions, utilising specific pharmaceutically acceptable carriers, that provide particular advantages with respect to compositions or carriers known from the prior art.

In particular, the inventors have identified that microspheres made of polymers such as PLGA represent pharmaceutically acceptable carriers that provide beneficial properties when used in pharmaceutical compositions of the invention. The siRNA molecules of the invention, and optionally other constituents of the pharmaceutical composition, may be encapsulated within these microspheres.

Suitably microspheres, such as PLGA microspheres, for use in pharmaceutical compositions of the invention may have a diameter of between about 1 μm and 60 μm; preferably between about 2 μm and 50 μm; more preferably between about 2 μm and 10 μm; and particularly preferably such microspheres may have a diameter of between about 2 μm and 5 μm.

The inventors have found that PLGA microspheres having a diameter of between about 2 μm and 5 μm exhibit unexpected beneficial properties in the way in which they are phagocytosed by cells associated with the development of canine osteoarthritis. Such microspheres may also be referred to as microbeads. Phagocytosis of microspheres or microbeads such as PLGA microspheres or microbeads, associated with the siRNA molecules of the invention represents a preferred route by which the siRNA molecules may be introduced into cells where they can exert their biological activity, therapeutically reducing the transcription and expression of TNF and/or IL-1β, depending on the siRNA molecule(s) encapsulated in the PGLA microspheres.

As described further in the Experimental Results section below, PLGA microspheres having a diameter of between approximately 2 μm and 5 μm were very effectively phagocytosed by DH82 cells, which provide an in vitro model of canine macrophages, and incorporated into the cells. Microspheres having diameters within this size range were incorporated into cells in greater numbers than larger microspheres. The larger number of microspheres incorporated in cells provides important advantages in terms of even distribution and dosing of the drug encapsulated in the microspheres (since variations in the numbers of microspheres, and hence drug payload, introduced into cells are reduced).

Methods of producing microspheres described elsewhere in the present specification provide high encapsulation efficiency, with about 70% of a drug being encapsulated within microspheres.

Drugs encapsulated in microspheres having a diameter below approximately 10 μm exhibit a relatively uniform distribution within the microspheres. While larger microspheres, such as those having diameters of approximately 100 μm or above, still enabled effective encapsulation of drugs, the drug molecules tended to aggregate in these microspheres and be distributed near the microspheres' surface. More uniform distribution of drug within the microspheres may be expected to provide improved properties in terms of the sustained and steady release of the drug within cells.

Indeed, the inventors have shown that microspheres provided effective control of release of the drugs encapsulated within them. As shown in the experimental results below, six weeks after administration to canine macrophages in vitro the microspheres still contained approximately 30 to 40% of their initial payload of encapsulated drug, demonstrating sustained release of the drug to the cells. Furthermore, these properties of sustained release were notably improved in microspheres having diameters between approximately 2 μm and 5 μm, as compared to larger microspheres, with the smaller microspheres exhibiting slower rates of degradation, and hence sustained drug release, as compared to microspheres having larger diameters.

Pharmaceutical compositions of the invention may include the siRNA of the invention as the only therapeutic agent. Alternatively, in suitable embodiments pharmaceutical compositions of the invention may comprise siRNA molecules of the invention in combination with one or more further therapeutic agents. In further alternative embodiments, pharmaceutical compositions of the invention, comprising siRNA molecules of the invention, may be formulated for use in combination with one or more further pharmaceutical compositions comprising one or more further therapeutic agents.

Pharmaceutical compositions of the invention comprising both siRNA molecules of SEQ ID NO. 2 (or variants based upon this sequence) and SEQ ID NO.7 (or variants based upon this sequence) are of particular utility. Such pharmaceutical compositions represent preferred agents to be employed in the medical uses and methods of treatment of the invention.

Methods of Treatment

The siRNA molecules of the invention are suitable for use in methods for the prevention and/or treatment of osteoarthritis in dogs. Such methods involve providing to a dog in need of such prevention and/or treatment a therapeutically effective amount of an siRNA molecule of the invention.

It will be appreciated that many of the factors applicable to the methods of treatment of the invention have already been considered in connection with the medical uses of the invention and the pharmaceutical compositions of the invention (which provide means by which the siRNA molecules of the invention may advantageously be provided in the methods of treatment). Accordingly, unless the context requires otherwise, it should be considered that all of the considerations set out above in connection with advantageous embodiments of the medical uses or pharmaceutical compositions of the invention are also applicable to the methods of treatment disclosed herein.

As referred to above, the methods of treatment of the invention involve the provision of siRNA molecules of the invention to a dog requiring prevention and/or treatment of osteoarthritis. The siRNA molecules may be provided by means of administering the nucleic acid to the subject in need of such prevention and/or treatment. For example, the nucleic acid may be administered in the form of a pharmaceutical composition of the invention such as a pharmaceutical composition comprising the nucleic acid in combination with PLGA microspheres, optionally be local injection of the pharmaceutical composition into a joint requiring the prevention and/or treatment of osteoarthritis.

Alternatively, the siRNA molecules of the invention may be provided to the subject requiring prevention and/or treatment of osteoarthritis by providing to the subject a vector that encodes the siRNA molecules, with the requisite siRNA molecules being provided by expression of the vector in situ. Suitably, the vector may be a viral vector that encodes the siRNA molecules of the invention.

The methods of treatment of the invention may utilise siRNA molecules of the invention as the sole therapeutic agents for the prevention and/or treatment of canine osteoarthritis. Alternatively, siRNA molecules of the invention may be used in combination with one or more further therapeutic agents, suitably other therapeutic agents that are able to prevent and/or treat osteoarthritis.

The siRNA molecules of the invention may be formulated with the further therapeutic agent(s) so that both the siRNA molecules and the further therapeutic agents are administered together. Alternatively, the siRNA molecules of the invention and further therapeutic agents may be administered separately but as part of a combined therapy, for example, through administration of two (or more) pharmaceutical compositions, one of which incorporates the siRNA molecules of the invention, and the other a different therapeutic agent.

In embodiments where it is wished to use the siRNA molecules of the invention in a combination therapy the further therapeutic agent may be selected from conventional agents known to those skilled in the art for the prevention and/or treatment of osteoarthritis.

Suitably siRNA molecules of the invention may be used in combination with other siRNA molecules that silence cytokines involved in the development and/or propagation of osteoarthritis. Merely by way of example, the siRNA molecules of the present invention may be used in combination with each other. For example the siRNA molecules of the invention that silence TNF (SEQ ID NOs. 1, 2, and 3, or nucleic acids based upon these sequences) may be used in combination with the siRNA molecules of the invention that silence IL-1β (SEQ ID NOs. 7, 8, and 9, or nucleic acids based upon these sequences). Alternatively, siRNA molecules comprising, or sharing at least 90% identity with, SEQ ID NOs. 7, 8, or 9 may be used in combination with the siRNA molecules of SEQ ID NOs. 1, 2, or 3, or siRNA molecules sharing at least 90% identity with these sequences. The combination of TNF silencing siRNA molecules of the invention comprising SEQ ID NO. 2 (or variants thereof) and IL-1β silencing siRNA molecules comprising SEQ ID NO. 7 (or variants thereof) is of particular utility.

Merely by way of example, the use of the siRNA molecules of the invention SEQ ID NOs. 1, 2, 3, 7, 8, or 9 in combination with other sequences of the invention (or sharing at least 90% identity with SEQ ID NOs. 1, 2, 3, 7, 8, or 9) may be achieved through the use of pharmaceutical compositions comprising both an siRNA molecule of the invention that silences TNF and an siRNA molecule of the invention that silences IL-1β. The siRNA molecules may be encapsulated in a microsphere, in the manner described further herein. In a suitable embodiment, both forms of siRNA molecules may be encapsulated in the same microspheres. In an alternative embodiment a suitable composition may comprise a mixture of microspheres, each containing a single form of siRNA.

The invention will now be further described with reference to the Experimental Results set out below, and to the accompanying Figures in which:

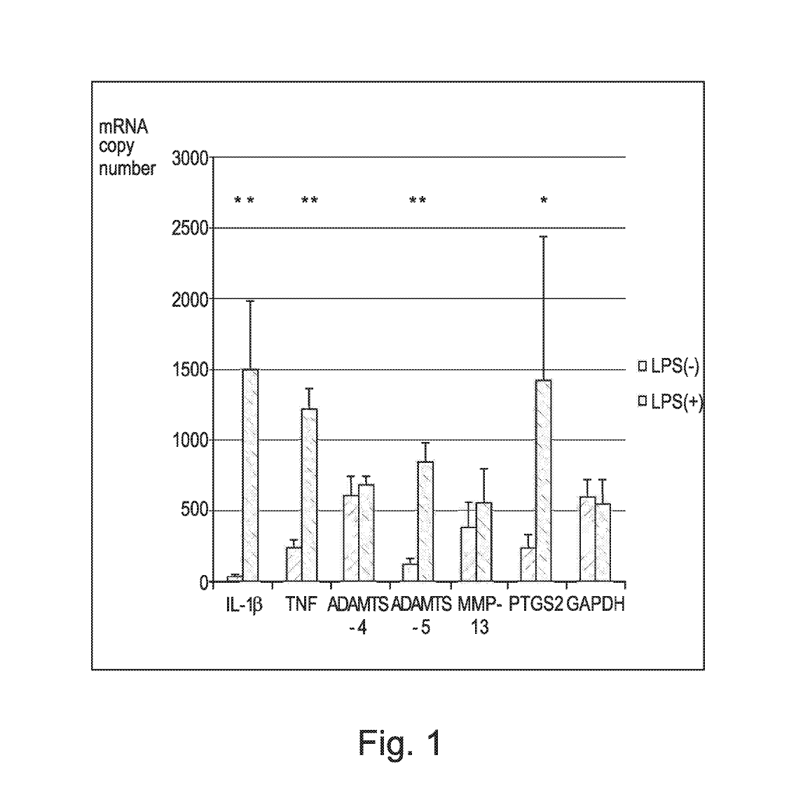

FIG. 1 shows mRNA expression (n=6 wells) for a number of genes associated with inflammation and osteoarthritis in DH82 cells with and without 24 hr incubation with lipopolysaccharide (*, p<0.05; **, p<0.001).

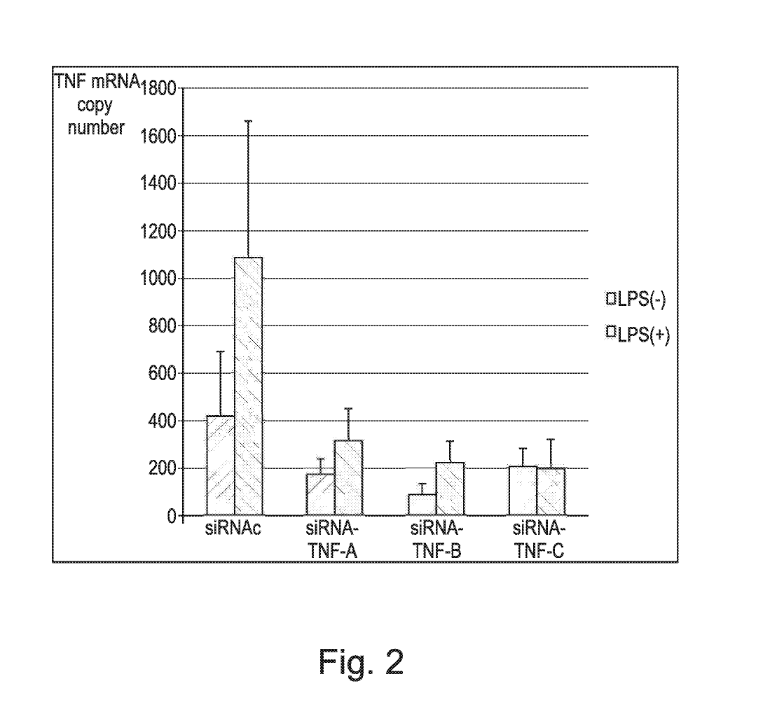

FIG. 2 shows mRNA expression (n=6 wells) for TNF in DH82 cells following transfection with one of three siRNA molecules targeted at canine TNF (nomenclature as set out in Table 4) or a generic scrambled siRNA molecule as negative control. Cells were incubated with or without lipopolysaccrahide to activate DH82 cells for 24 hr prior to RNA extraction.

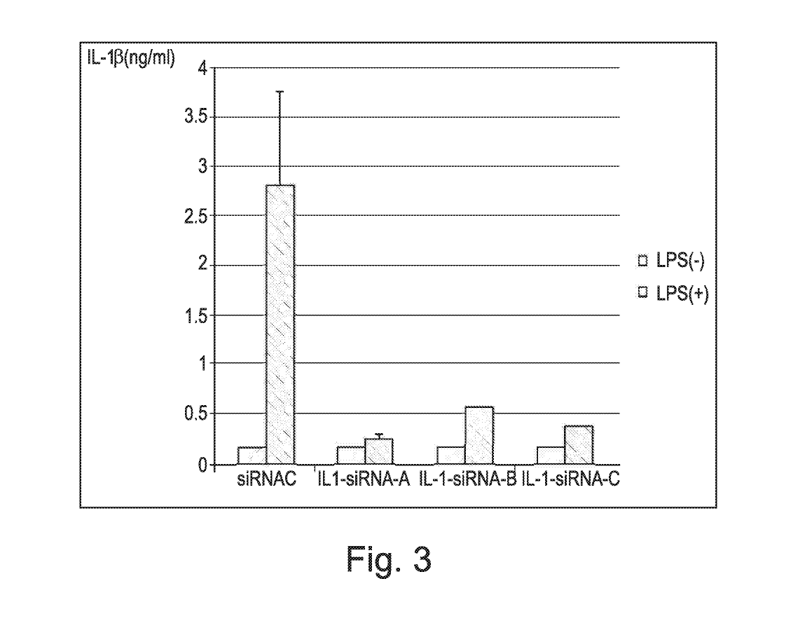

FIG. 3 shows mRNA expression (n=6 wells) for IL-1 in DH82 cells following transfection with siRNA-IL-1-A (SEQ ID NO. 1) or a generic scrambled siRNA molecule as negative control. Cells were incubated with or without lipopolysaccharide to activate DH82 cells for 24 hr prior to RNA extraction.

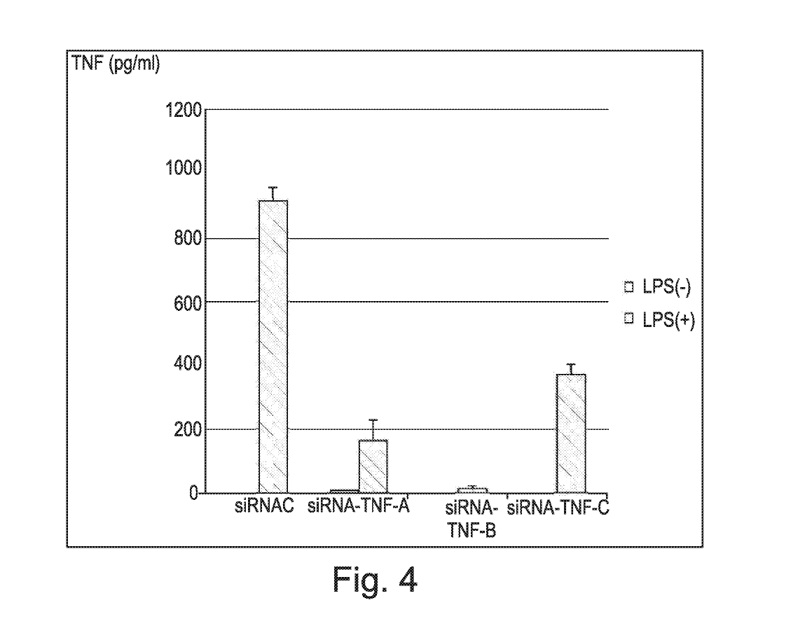

FIG. 4 shows TNF protein concentrations in culture media (n=6 wells) following transfection of DH82 cells with one of three siRNA molecules targeted at canine TNF (nomenclature as set out in Table 4) or a generic scrambled siRNA molecule as negative control. Cells were incubated with or without lipopolysaccharide to activate DH82 cells for 24 hr prior to harvesting of culture media.

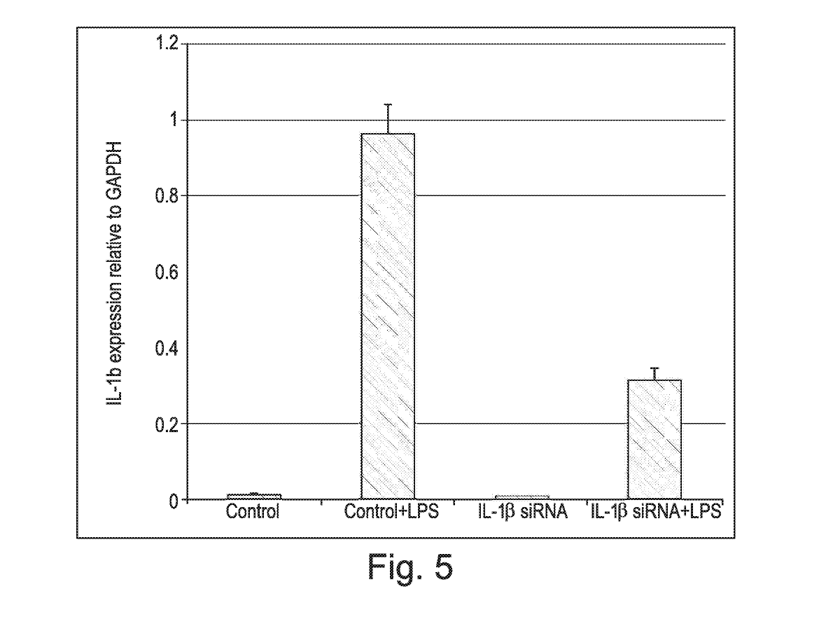

FIG. 5 shows IL-1 protein concentrations in culture media (n=6 wells) following transfection of DH82 cells with one of three siRNA molecules targeted at canine IL-1 or a generic scrambled siRNA molecule as negative control. Cells were incubated with or without lipopolysaccharide to activate DH82 cells for 24 hr prior to harvesting of culture media.

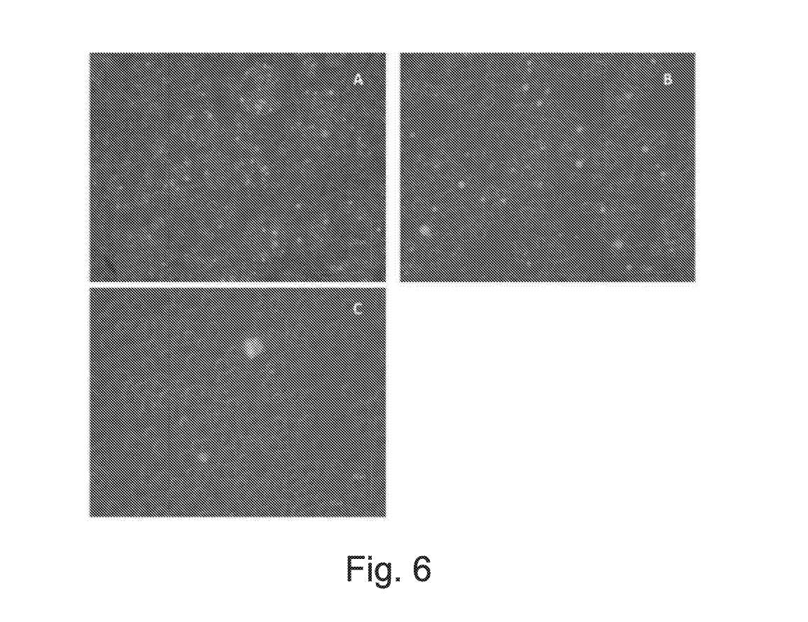

FIG. 6 shows microspheres loaded with fluorescent dyes as drug proxies after 1 week in culture.

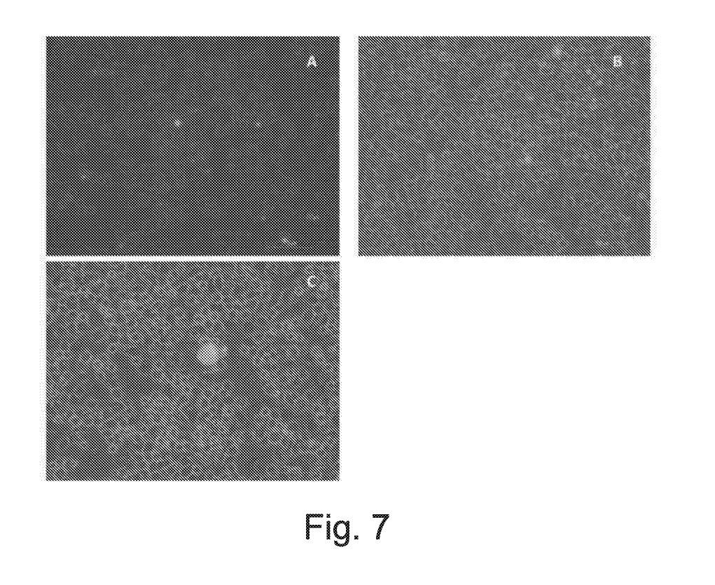

FIG. 7 shows microspheres loaded with fluorescent dyes as drug proxies after 3 weeks in culture.

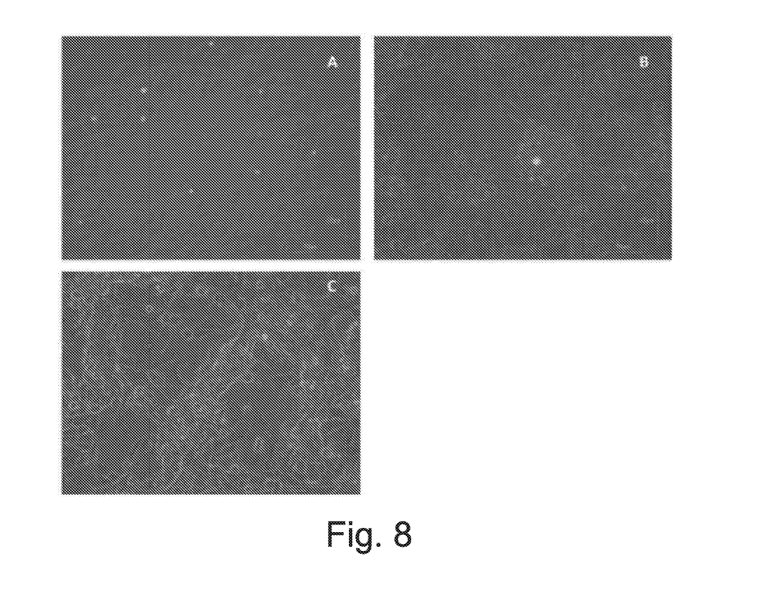

FIG. 8 shows microspheres loaded with fluorescent dyes as drug proxies after 6 weeks in culture.

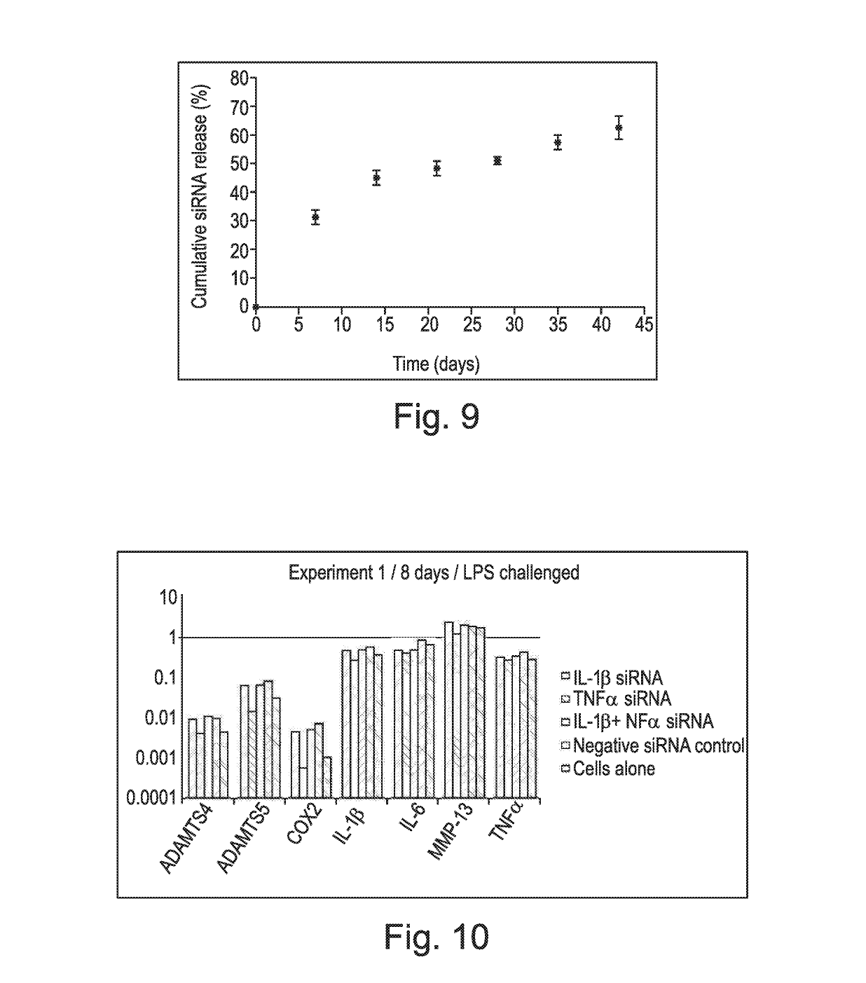

FIG. 9 shows cumulative siRNA release profiles from PLGA microspheres in PBS at pH=7.4 at 37° C.

FIG. 10 shows expression of key DH82 target genes when cultured with two 24 hour LPS challenges (days two and six) for a duration of eight days. Cells were cultured with and without the siRNA loaded and negative control loaded microspheres. Analysis is by quantitative RT-PCR and displayed with a logarithmic y-axis. All genes have been normalised against the expression of the house keeping gene GAPDH.

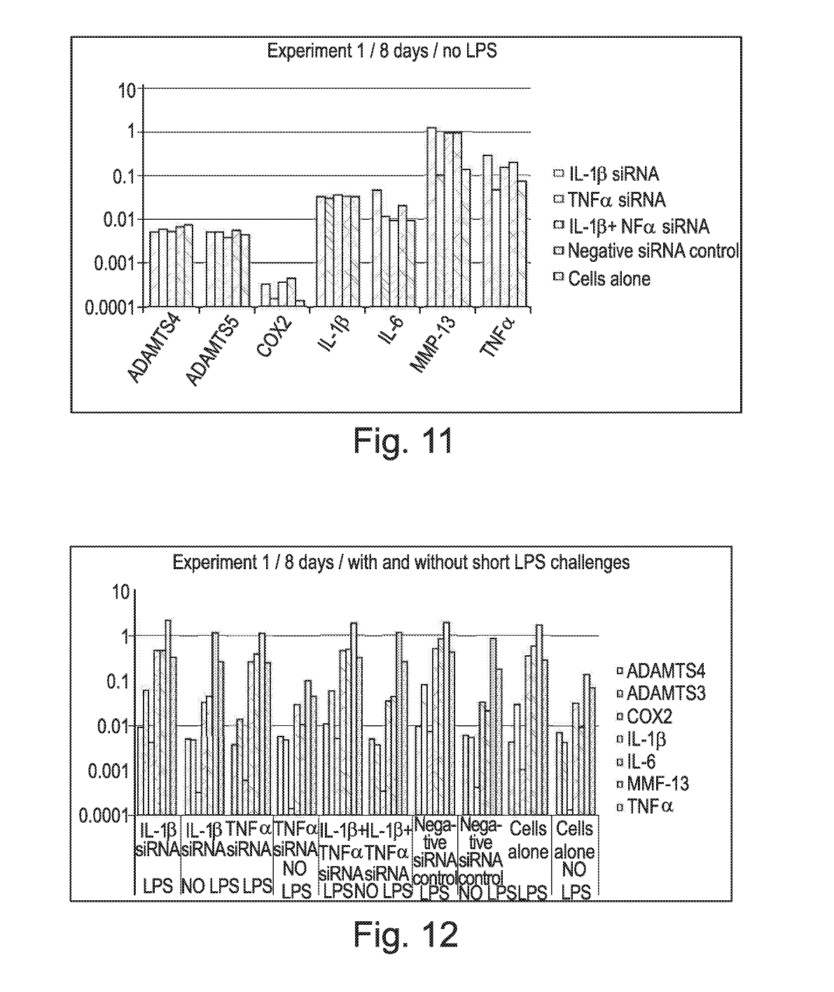

FIG. 11 shows expression of key DH82 target genes when cultured without LPS challenges for a duration of eight days. Cells were cultured with and without the siRNA loaded and negative control loaded microspheres. Analysis is by quantitative RT-PCR and displayed with a logarithmic y-axis. All genes have been normalised against the expression of the house keeping gene GAPDH.

FIG. 12 shows expression of key DH82 target genes when cultured with and without LPS challenges (days two and six) for a duration of eight days. Cells were cultured with and without the siRNA loaded and negative control loaded microspheres. Analysis is by quantitative RT-PCR and displayed with a logarithmic y-axis. All genes have been normalised against the expression of the house keeping gene GAPDH.

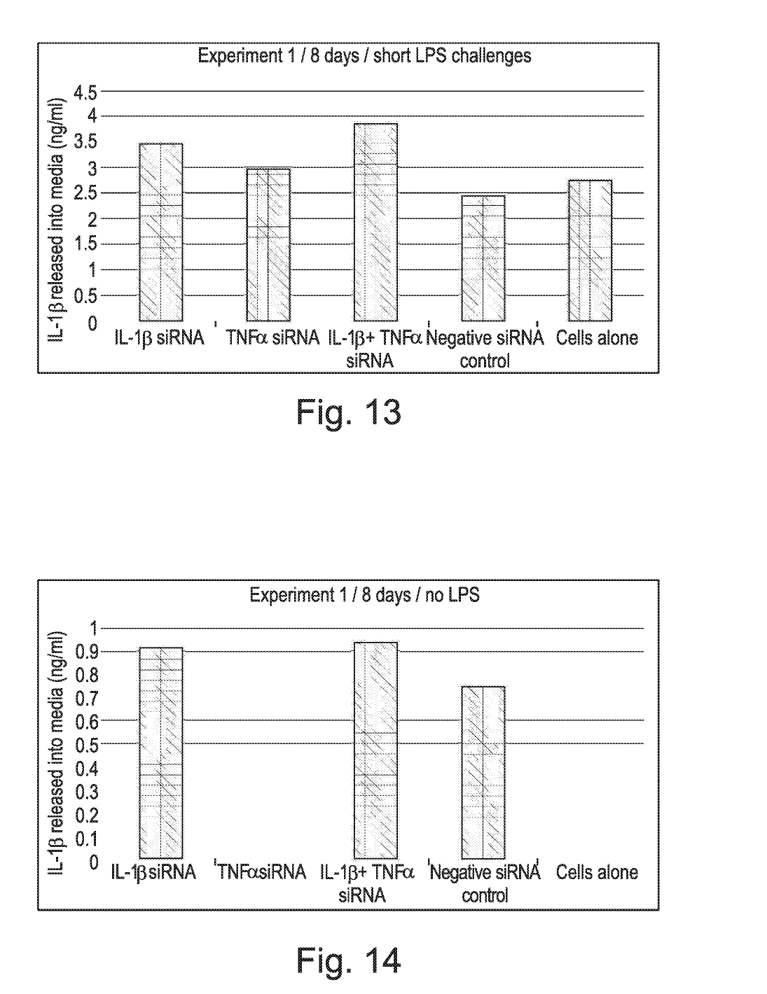

FIG. 13 shows IL-1β protein released into the culture media from DH82 cells when cultured with two 24 hour LPS challenges (days two and six) for a duration of eight days. Cells were cultured with and without the siRNA loaded and negative control loaded microspheres.

FIG. 14 shows IL-1β protein released into the culture media from DH82 cells when cultured without LPS for eight days with and without the siRNA loaded and negative control loaded microspheres.

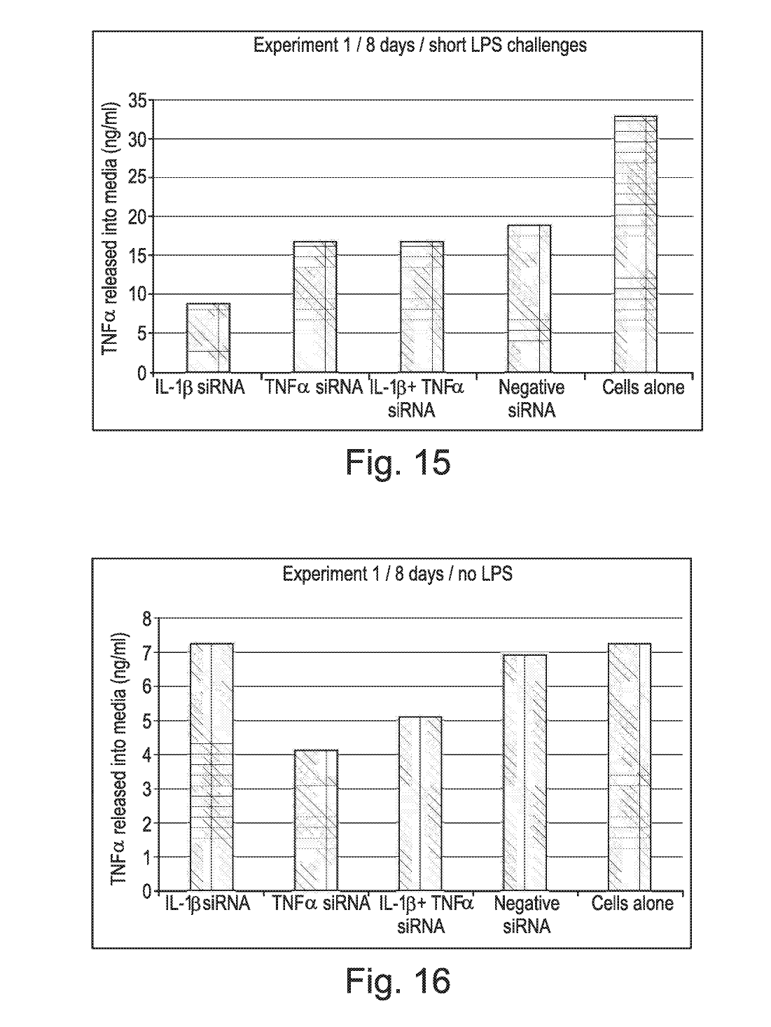

FIG. 15 shows TNF protein released into the culture media from DH82 cells when cultured with two 24 hour LPS challenges (days two and six) for a duration of eight days. Cells were cultured with and without the siRNA loaded and negative control loaded microspheres.

FIG. 16 shows TNF protein released into the culture media from DH82 cells when cultured without LPS for eight days with and without the siRNA loaded and negative control loaded microspheres.

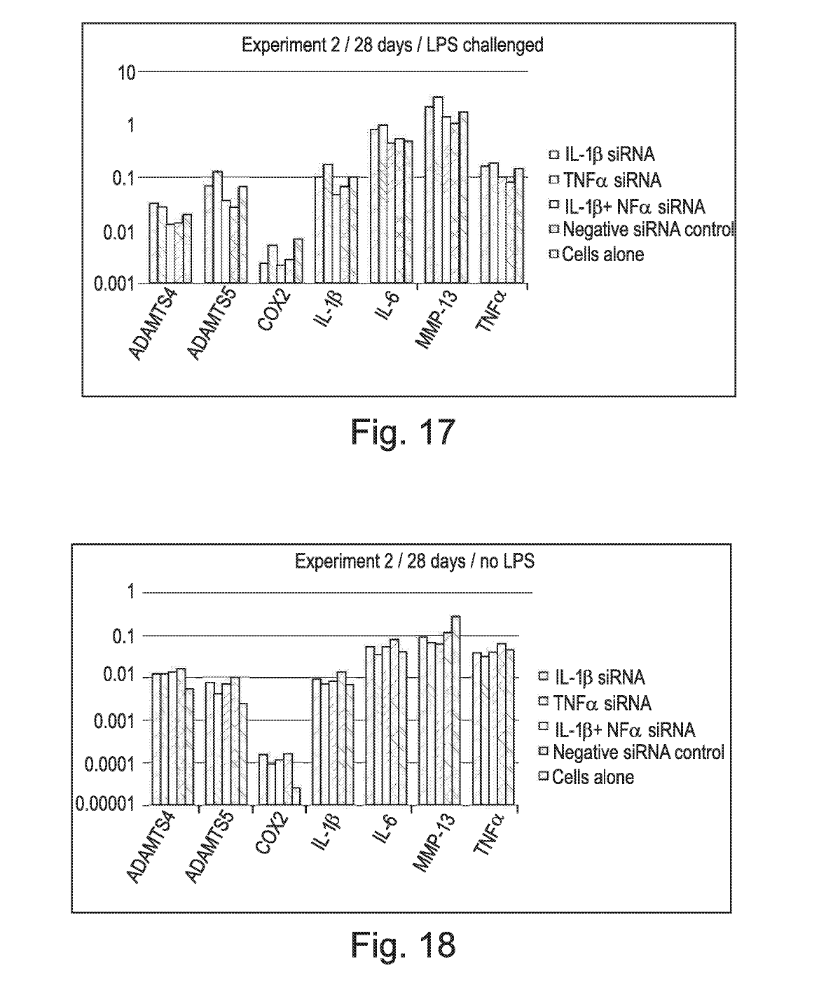

FIG. 17 shows expression of key DH82 target genes when cultured with two 24 hour LPS challenges per week for duration of four weeks. Cells were cultured with and without the siRNA loaded and negative control loaded microspheres. Analysis is by quantitative RT-PCR and displayed with a logarithmic y-axis. All genes have been normalised against the expression of the house keeping gene GAPDH.

FIG. 18 shows expression of key DH82 target genes when cultured without LPS for duration of four weeks. Cells were cultured with and without the siRNA loaded and negative control loaded microspheres. Analysis is by quantitative RT-PCR and displayed with a logarithmic y-axis. All genes have been normalised against the expression of the house keeping gene GAPDH.

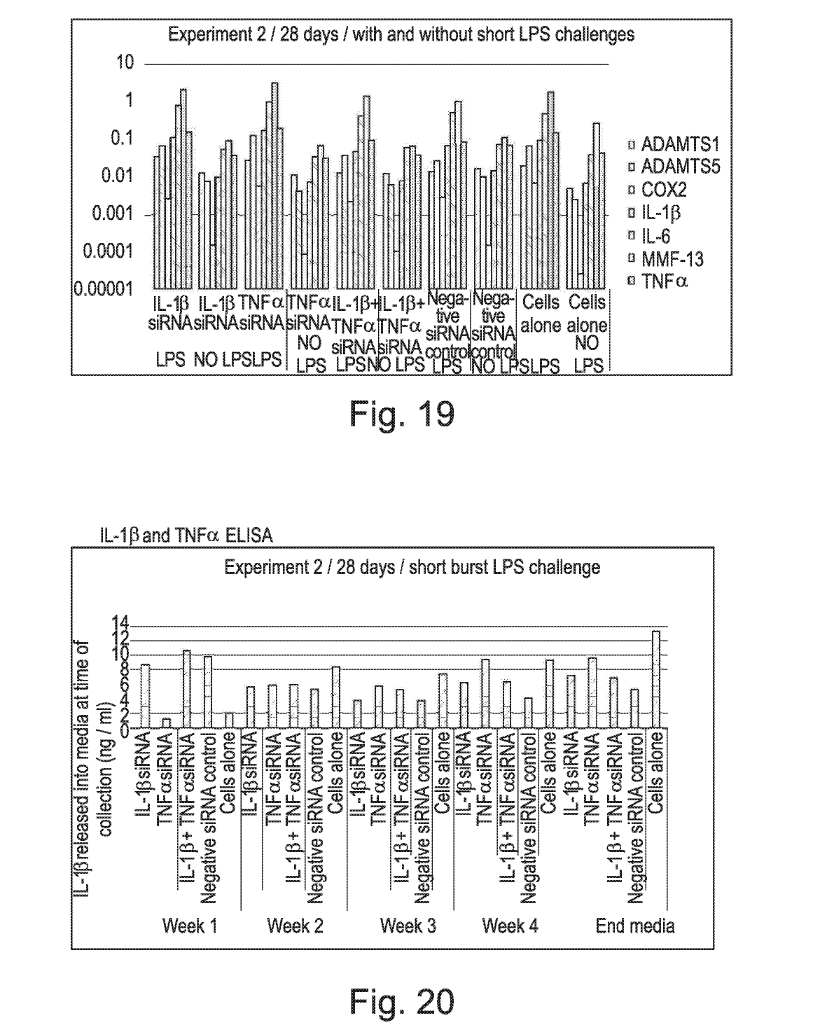

FIG. 19 shows expression of key DH82 target genes when cultured with and without LPS challenges (two per week) for duration of 28 days. Cells were cultured with and without the siRNA loaded and negative control loaded microspheres. Analysis is by quantitative RT-PCR and displayed with a logarithmic y-axis. All genes have been normalised against the expression of the house keeping gene GAPDH.

FIG. 20 shows IL-1β protein released into the culture media from DH82 cells when cultured with two 24 hour LPS challenges per week for duration of 28 days. Cells were cultured with and without the siRNA loaded and negative control loaded microspheres.

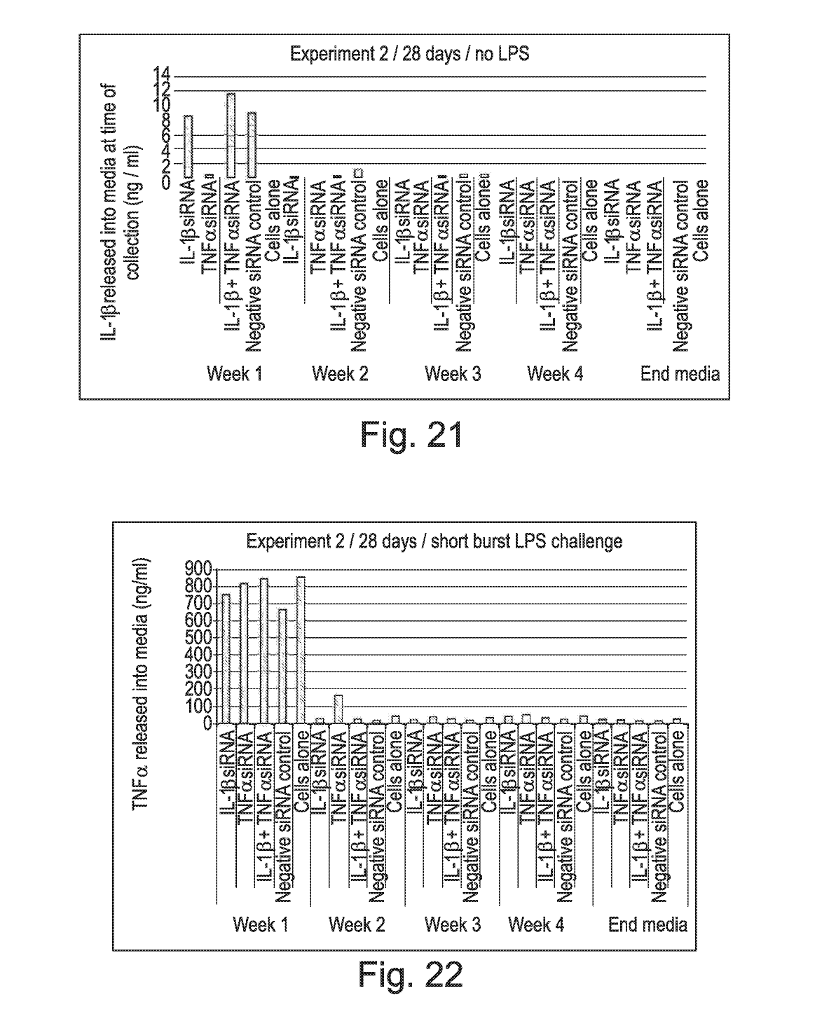

FIG. 21 shows IL-1β protein released into the culture media from DH82 cells when cultured without LPS for duration of 28 days. Cells were cultured with and without the siRNA loaded and negative control loaded microspheres.

FIG. 22 shows TNF protein released into the culture media from DH82 cells when cultured with two 24 hour LPS challenges per week for duration of 28 days. Cells were cultured with and without the siRNA loaded and negative control loaded microspheres.

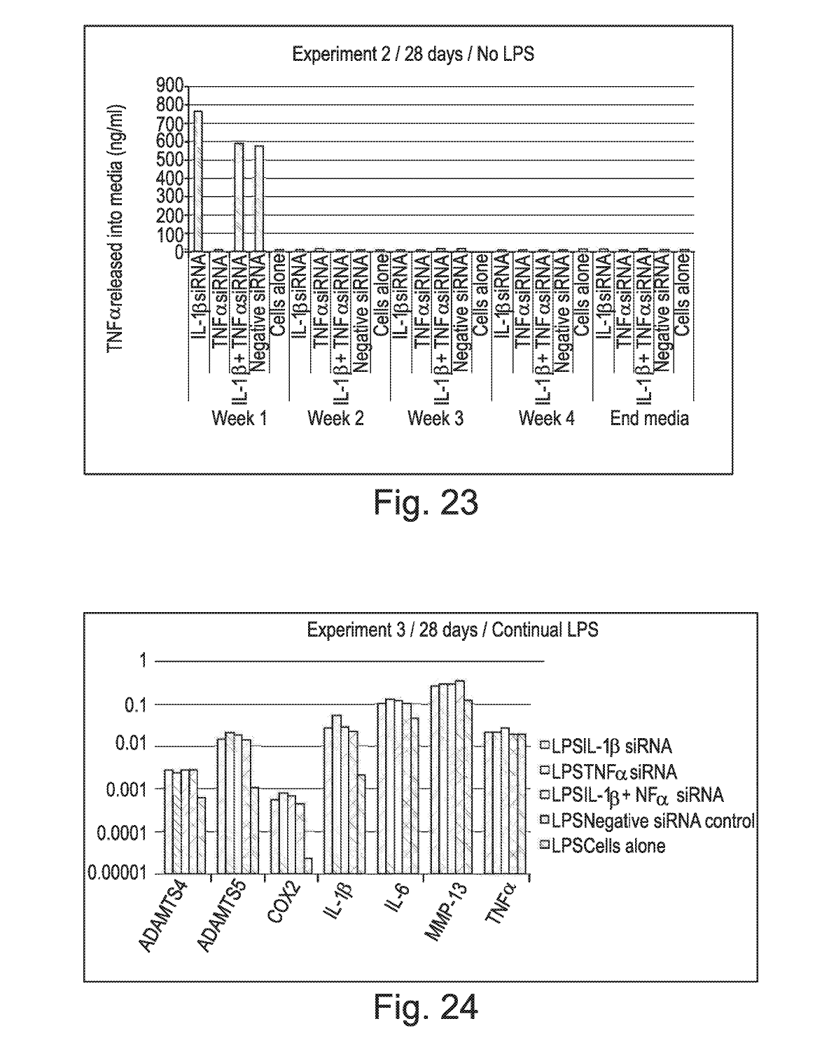

FIG. 23 shows TNF protein released into the culture media from DH82 cells when cultured without LPS for duration of 28 days. Cells were cultured with and without the siRNA loaded and negative control loaded microspheres.

FIG. 24 shows expression of key DH82 target genes when cultured with continual LPS stimulation for duration of four weeks. Cells were cultured with and without the siRNA loaded and negative control loaded microspheres. Analysis is by quantitative RT-PCR and displayed with a logarithmic y-axis. All genes have been normalised against the expression of the house keeping gene GAPDH.

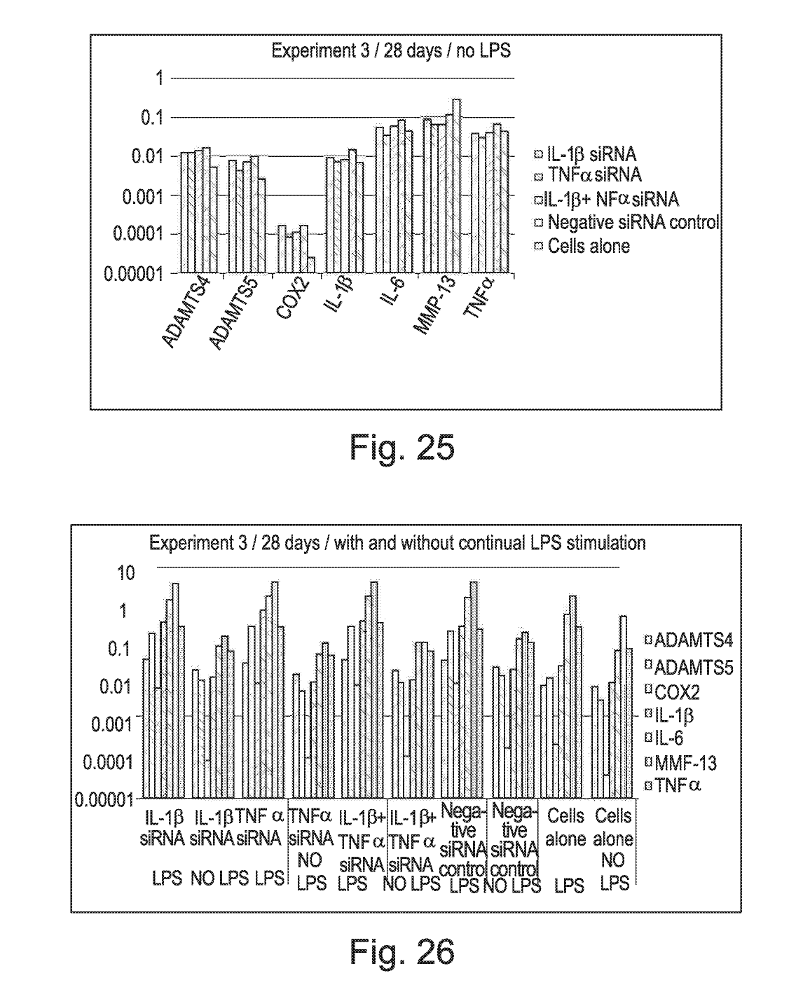

FIG. 25 shows expression of key DH82 target genes when cultured without LPS stimulation for duration of four weeks. Cells were cultured with and without the siRNA loaded and negative control loaded microspheres. Analysis is by quantitative RT-PCR and displayed with a logarithmic y-axis. All genes have been normalised against the expression of the house keeping gene GAPDH.

FIG. 26 shows expression of key DH82 target genes when cultured with and without continual LPS stimulation for duration of 28 days. Cells were cultured with and without the siRNA loaded and negative control loaded microspheres. Analysis is by quantitative RT-PCR and displayed with a logarithmic y-axis. All genes have been normalised against the expression of the house keeping gene GAPDH.

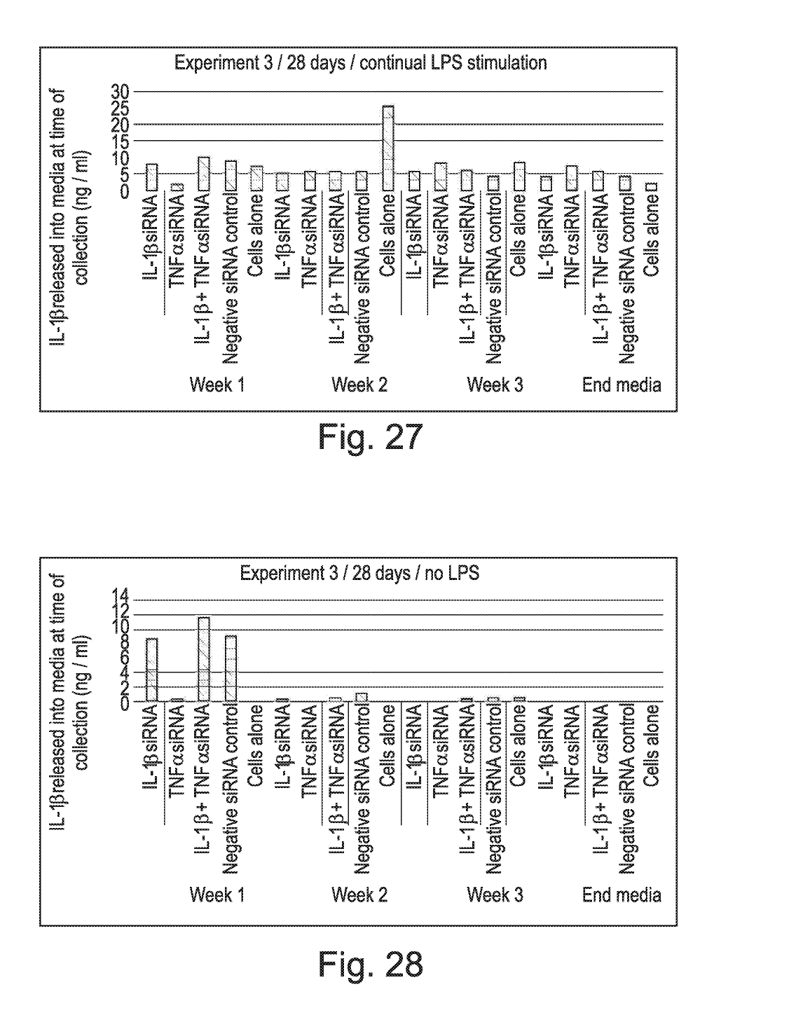

FIG. 27 shows IL-1β protein released into the culture media from DH82 cells when cultured with continual LPS stimulation for duration of 28 days. Cells were cultured with and without the siRNA loaded and negative control loaded microspheres.

FIG. 28 shows IL-1β protein released into the culture media from DH82 cells when cultured without continual LPS stimulation for duration of 28 days. Cells were cultured with and without the siRNA loaded and negative control loaded microspheres.

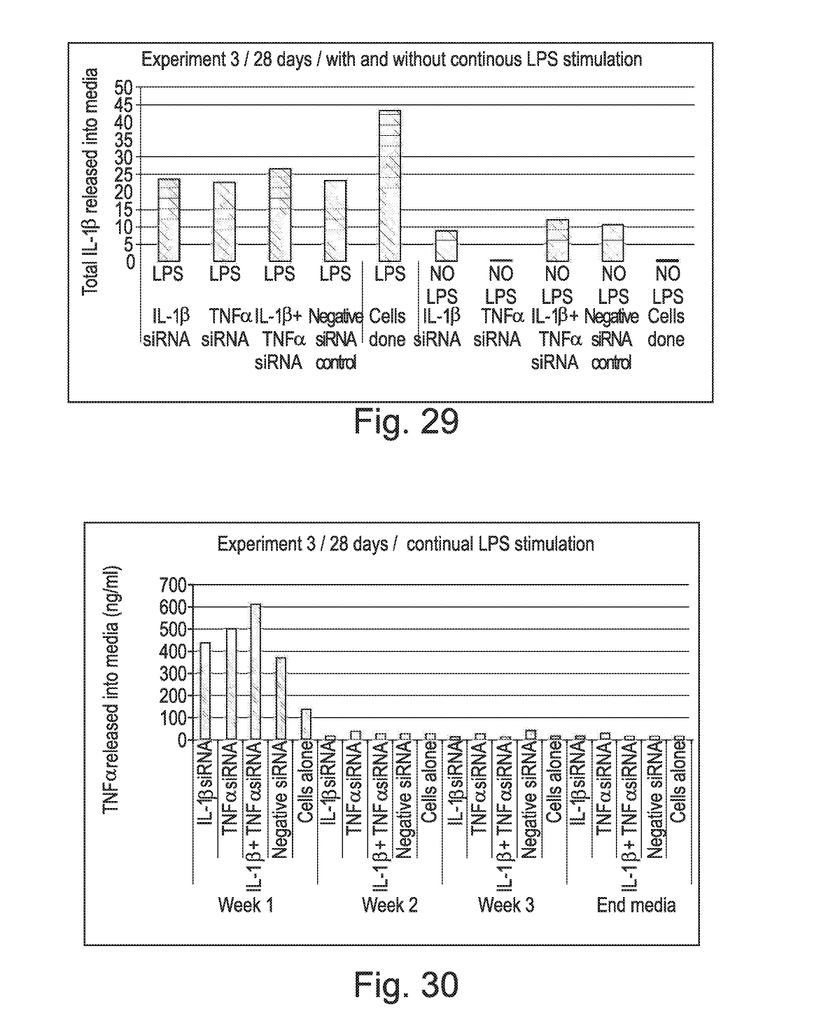

FIG. 29 shows Cumulative IL-1β protein released into the culture media from DH82 cells when cultured without continual LPS stimulation for duration of 28 days. Cells were cultured with and without the siRNA loaded and negative control loaded microspheres.

FIG. 30 shows TNF protein released into the culture media from DH82 cells when cultured with continual LPS stimulation for duration of 28 days. Cells were cultured with and without the siRNA loaded and negative control loaded microspheres.

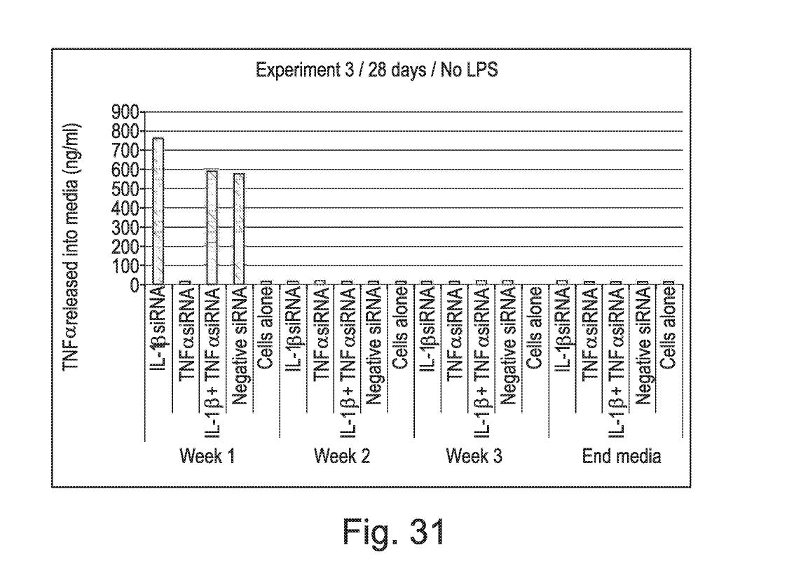

FIG. 31 shows TNF protein released into the culture media from DH82 cells when cultured without continual LPS stimulation for duration of 28 days. Cells were cultured with and without the siRNA loaded and negative control loaded microspheres.

### EXPERIMENTAL RESULTS

### Study 1

Experimental Design

Cell Culture Systems

Canine monocyte-macrophage DH82 cells (Wellman et al., 1988) (Health Protection Agency Culture Collections, Salisbury, UK) were maintained in 75 cm² flasks of Eagle's minimal essential medium (EMEM) (Invitrogen, Australia) supplemented with 2 mM glutamine, 1% non-essential amino acids and 15% foetal calf serum (FCS; Trace Biosciences Pty. Ltd., Castle Hill, NSW, Australia). Cells for transfection studies were plated out (n=6) in 24-well clear bottom cell culture plates at 1.5×10³ cells in 500 μl of culture medium for 24 hours before medium was changed to 200 μl OPTI-mem® (Invitrogen) reduced serum medium. Cells were then transfected with each one of three siRNAs at 20 nmol targeted at canine TNF (SEQ ID NOs. 1 to 3 set out in Table 4) or targeted at canine IL-1β (SEQ ID NOs 7 to 9 set out in Table 5); a scrambled generic siRNA was used as a negative control in all transfection experiments. Lipofectamine 2000™ (Invitrogen) was used as a transfection agent according to the manufacturer's instructions and cells were incubated for 4 h before the addition to each well of 300 μl EMEM supplemented with 20% FBS and overnight incubation. The media were then changed to EMEM supplemented with 2 mM glutamine, 1% non-essential amino acids and 15% FCS with or without lipopolysaccharide (LPS) (Sigma-Aldrich, Castle Hill, NSW, Australia) for 24 hours.

Primary cultures of canine synovial cells were established from synovial tissue removed as clinical waste during canine orthopaedic surgical procedures at University of Sydney Veterinary School. Briefly, synovial tissue was finely minced, washed, and incubated in 0.1% trypsin solution followed by incubation in Dulbecco's modified Eagles medium (DMEM) containing 10% foetal bovine serum (FBS) and 0.2% collagenase. Isolated synoviocytes were washed three times and then plated and grown to subconfluency before use in experiments. Synovial cell cultures were transfected with the siRNA of SEQ ID NO. 2 under similar conditions to those described for DH82 cells above. This siRNA molecule of the invention was selected since it was the best-performing siRNA in terms of its ability to silence TNF (as determined by experiments in DH82 cells described above).

Quantitative Real-Time Gene Expression Assays

RNA was extracted from cell cultures using TRIzol (1 ml/100 mg tissue; Invitrogen, Melbourne, Victoria, Australia), chloroform, and RNeasy spin columns (Qiagen, Doncaster, Victoria, Australia), according to the recommendations of the manufacturer. An on-column DNase step (Qiagen) was included to remove any potential genomic DNA contamination. RNA (500 ng) from each sample was reverse-transcribed (Omniscript; Qiagen). Real-time PCR for selected genes was performed on the resulting cDNA in a Rotorgene 6000 (Corbett Life Science, NSW, Australia) using Immomix (2× dilution; Bioline, Alexandria, New South Wales, Australia), SYBR Green I (10,000× dilution; Cambrex Bioscience), and 0.3 μM canine-specific primers (Table 1). Primers with known specificity to canine genes were either previously published or designed using MacVector software, version 7.2.2 (Accelrys, San Diego, Calif.) (Table 1) Standard curves (4-fold dilutions of canine DH82 cDNA) were included, and a relative copy number was determined for each gene of interest. Melt curves were obtained to check for single products.

ELISA Assays for Canine TNF/IL-1β

Culture media were assayed for canine TNF/IL-1β protein using commercially-available kits (Canine TNF Quantikine, R&D Systems; Canine IL-1β VetSet, Kingfisher Biotech) as recommended in the manufacturer's instructions

Formulation and Fabrication of PLGA Microspheres

Drug loaded microspheres were prepared using the water-in-oil-in-water (W1/O/W2) solvent-evaporation technique. Briefly, PLGA (65:35, Mw=40,000-75,000) was dissolved into dichloromethane at 100 mg/ml, 10 mg/ml and 4 mg/ml. FD4 (Fluorescein isothiocyanate-dextran, Mw=3,000-5,000) and FD10 (Fluorescein isothiocyanate-dextran, Mw=10,000), fluorescent agents, used as model drugs in this study due to their ease of detection within microspheres and cells, were dissolved in pure water at 100 mg/ml. FD4/FD10 solution as an inner-water phase was poured into the PLGA solution (10% w/w). Water-in-oil (W1/O) emulsion was prepared with a micro-homogenizer in a 25 ml flask for 30 seconds at 4° C. Prepared emulsion (W1/O) was subsequently added to 100 ml 1% PVA (Mw=13,000-23,000) solution. Then the emulsion was stirred (1000 rpm) to obtain W1/O/W2 emulsion for 30 minutes. The prepared emulsion was poured into 100 ml 0.1% PVA solution to prevent coagulation and evaporate the solvent under stirring at 400 rpm for 4 hours at room temperature. The microspheres were collected and washed 4 times using centrifugation. The collected microspheres were freeze-dried for 24 hours and then stored at −20° C. before use.

In Vitro Studies with Drug Loaded Microspheres

The following study was undertaken t Three sizes of microsphere tested: small (2 to 5 μm), medium (5-50 μm) and large (50-100 μm). Each size was tested in duplicate and was labelled with either FD4 or FD10 fluorescent label.

DH82 cells seeded into 6 well plates at 5×10³ cells/cm₂ and cultured in standard DH82 growth media (15% FSB and 1% NEAA). 2 mg of the microspheres to be tested were added per well. The cells and microspheres were then incubated for 7 days with no intermediate media changes.

After this time, the media was harvested. To maintain the health of the cell populations, it was necessary to passage the cells and re-seed at a diluted factor. All of the cells were retained, thus the designated growth area for each microsphere/cell co-culture expanded.

The experiment was maintained as described above for 6 weeks. The media was harvested every 7 days at which time the cells were passaged and transferred into larger culture plates. All of the cells were retained. Pictures were taken at each time point to test the ability of the DH82 cells to maintain incorporation of the microspheres over a prolonged time period, and representative images are shown in FIGS. 6 to 8.

Results

Response of DH82 Cells to Activation with LPS

DH82 cells showed significant upregulation of the majority of the genes investigated in response to stimulation with LPS. Notably, in contrast to the DH82 data sheet, we demonstrated that DH82 cells express the IL-1β gene and this is upregulated in response to LPS. We also demonstrated expression of MMP-13, ADAMTS-4, ADAMTS-5 and PTSG2 and subsequent upregulation of genes for MMP-13, ADAMTS-5 and PTSG2 (FIG. 1).

Knock-Down in Expression of Canine Candidate Genes

All six siRNA molecules of the invention induced significant knockdown of their target genes. Of the three siRNA molecules of the invention targeted against TNF, molecule TNF-siRNA-B′ (SEQ ID NO. 2) was judged to have the most efficient knockdown with a 78% reduction in TNF gene expression in inactivated DH82 cells and a 80% reduction in TNF gene expression compared to LPS-stimulated controls (FIG. 2). Additionally, of the three siRNA molecules of the invention targeted against IL-1β, ‘IL-1β-siRNA-A’ (SEQ ID NO. 7) was the most efficient in terms of its ability to reduce gene expression compared to LPS-stimulated controls (FIG. 3). No off-target effects were noted in that there were no significant effects on the expression of other genes assessed (GAPDH, ADAMTS-4, ADAMTS-5, MMP-13, PTSG2).

Measurement of TNF protein with ELISA indicated significant suppression of cytokine production following siRNA transfection (FIG. 4). Using siRNA-TNF-‘B’ (SEQ ID NO. 2), there was no TNF detected in inactivated DH82 cell culture medium and following activation with LPS we observed a 99% reduction in TNF protein concentration compared to scrambled siRNA negative control (FIG. 4).

Similarly, measurement of IL-1β protein with ELISA indicated significant suppression of cytokine production following siRNA transfection (FIG. 5). Using ‘IL-1β-siRNA-A’ (SEQ ID NO. 7), there was no IL-1β detected in inactivated DH82 cell culture medium, but following activation with LPS we observed a 91% reduction in IL-1β protein concentration with ‘siRNA-IL-1β’ compared to scrambled siRNA negative control (FIG. 5).

These results demonstrate the advantages that may be achieved by medical uses, methods of treatment, or pharmaceutical compositions of the invention in which siRNA molecules of both SEQ ID NO.2 (or variants thereof) and SEQ ID NO.7 (or variants thereof) are employed.

Results of In Vitro Microsphere Studies

A first observation by the inventors was that, since both florescent labels are water soluble, the intensity of both decreased over time. However FD10 labelling was more effective than FD4.

Results after 1 Week of Culture (FIG. 6)

By visual examination, it was evident that the small microspheres were incorporated effectively into the DH82 cells. Numerous microspheres could be phagocytosed by individual cells. The medium microspheres were also incorporated into the cells although this was predominantly limited to one or two per cell. The very large microspheres i.e. the ones on the top end of the size range, appeared unable to be fully phagocytosed by the cells.

Results after 3 Week of Culture (FIG. 7)

By week 3, the microspheres have been diluted due to passaging the cells to maintain their health, thus they have been distributed over a greater growth area. Therefore upon visual examination, there appears to be fewer microspheres incorporated into the cells compared to total cell number. However, it should be borne in mind that unincorporated microspheres will have been lost in the passaging process.

At this point, small and medium microspheres could still be detected within DH82 cells. Very few large microspheres could be detected and the ones that were identified did not appear to be fully phagocytosed.

Results after 6 Weeks of Culture (FIG. 8)

At this time, the cells appeared less healthy with a large number having detached from the plastic. Small and medium microspheres were still detected in the cells. Large microspheres were also detected although they were much smaller than the large microspheres identified after week 1.

Investigation of TNF and IL-1β Expression

The extent to which treatment with drug loaded microspheres induced an inflammatory reaction by cultured cells was investigated using commercially available canine specific ELISA kits to calculate the levels of TNF and IL-1β in the cell culture media that had been collected at each time point.

TNF was only detected in the media collected in week one and only where small microspheres (both FD4 and FD10) had been added. FD4 small microspheres released 91.5 pg/ml from the DH82 cells and FD10 small microspheres released 85 pg/ml.

IL-1β was detected at low levels in media collected from weeks 1 and 2 only and it was only released from cells where the small microspheres had been added (both FD4 and FD10). Week 1 FD4 small microsphere: 0.33 ng/ml, week 1 FD10 small microsphere: 0.34 ng/ml, week 2 FD4 small microsphere: 0.03 ng/ml, week 2 FD10 small microsphere: 0.01 ng/ml.

Conclusions Regarding Drug Loaded Microspheres

Small (2 to 5 μm) and medium (5-50 μm) microspheres were effectively incorporated into DH82 cells. A vast number of small microspheres could be phagocytosed into one cell, whereas this was limited to only one or two of the medium microspheres. This is an important consideration for calculating the siRNA dosage per cell, and indicates a significant advantage on the part of pharmaceutical formulations comprising smaller microspheres.

Both small and medium microspheres were still evident within cells following 6 weeks of culture.

The large microspheres ranged in size between 50 and 100 μm. The microspheres that were at the larger end of this range were unable to be fully phagocytosed by the cells and therefore many were lost during the weekly passaging. By week 6, large microspheres were still detected although they were much smaller than the microspheres identified in week 1. This could be because the large microspheres had degraded or it could be due to the fact that the larger ones had been washed away and only the smaller ones remained i.e. the ones whose smaller size allowed them to be incorporated into the cells.

Addition of the small microspheres elicited a transient inflammatory response from the cells, as evidenced by TNF and IL-1β release. This was only found in the early stages on the experiment. This could be due to the fact that a vast number of small microspheres were phagocytosed compared to the medium and large microspheres which were more limited by their size. It is expected that when microspheres encapsulating the siRNA molecules of the invention are used, the gene silencing effects of the siRNA molecules will block this inflammatory response.

### Study 2

SiRNA Loading Efficiency and Release Studies

To determine the amount of siRNA encapsulated, the freeze-dried 5 mg microspheres were dissolved in 2 ml of dichloromethane in a capped glass vial and shaken for 3 hours. 2 ml of 0.1M PBS at pH=7.4 was added and mixed vigorously for 2×30 seconds and then centrifuged at 3600 rpm at 4° C. for 30 min. The aqueous phase was collected and analysed for siRNA concentration by UV spectrophotometry at 260 nm (Eppendorf®, BioPhotometer, Germany).

To analyse the in vitro release of siRNA from microspheres, 20 mg PLGA microspheres were suspended in 2 ml of 0.1M phosphate buffer saline (PBS) pH=7.4 and stored at 37° C. under moderate horizontal agitation. At predetermined time intervals (every 7 days) 1.5 ml of the medium was collected following centrifugation and then replaced with 1.5 ml PBS. The siRNA concentrations were also analysed by UV spectrophotometry at 260 nm (Eppendorf®, BioPhotometer, Germany).

Results

SiRNA Loading Efficiency and Controlled-Release

The loading efficiency of siRNA in PLGA microspheres was 68.74±7.90%. The amount of siRNAs loaded in PLGA microspheres was 0.19±0.05 nmol/mg of microspheres.

FIG. 9 shows siRNA release profiles from PLGA microspheres in PBS at pH=7.4 at 37° C. The results indicated that siRNA rapidly released in first 2 weeks, SiRNA released about 45% in first 2 weeks; After 2 weeks, the release rate decreased, the cumulative siRNA release increased to 51% at week 4; after 5 weeks, the release rate increased further. However, only 63% of the siRNA loaded had released after 6 weeks.

### Study 3

In Vitro siRNA Loaded PLGA Microspheres on DH82 Cell Functional Assay

DH82 cells were thawed and seeded into T75 vented culture flasks. Media: MEM, 10% FBS, 100 U/ml penicillin, 100 μg/ml streptomycin, 500 ng/ml amphotericin B, 1% non-essential amino acids (NEAA) [all Invitrogen, Paisley, UK]. Cells were cultured overnight, passaged and then reseeded into new T75 culture flasks at 10⁶ cells/cm². To reduce the rate of cellular proliferation the FBS in the media was reduced to 5%. The cells were left for 1 hour to adhere and then used immediately for the following three experiments to investigate the in vitro effects of siRNA-loaded PLGA microspheres.

Experiment 1: 8 Day Experiment with 2×24 hr LPS Bursts.

Microspheres were added to the flasks at a concentration of 1 mg per 1 ml of media. Each flask contained 10 mls media, therefore a total of 10 mg of siRNA loaded microspheres were added per flask.

The cells were subjected to two LPS (Sigma) challenges (1 μg/ml) at day 2 and day 6, each lasting for 24 hours. Media was collected and replaced after each LPS challenge and at the completion of the experiment. As a control, loaded microspheres were also added to DH82 cells without subjection to LPS. For these cells, the media was collected and replaced with standard media at day 2 and 6 and collected at the completion of the experiment. An additional control involved DH82 cells alone both with and without LPS bursts.

At completion of the experiment the cells were preserved in Tri Reagent (Sigma) and stored at −80° C. to be used for RNA isolation.

### SUMMARY

| | |
|---|---|
| DH82 cells + IL siRNA | 2 × LPS BURSTS |
| DH82 cells + TNF siRNA | 2 × LPS BURSTS |
| DH82 cells + IL and TNF siRNA | 2 × LPS BURSTS |
| DH82 cells + negative control | 2 × LPS BURSTS |
| DH82 cells alone | 2 × LPS BURSTS |
| DH82 cells + IL siRNA | NO LPS BURSTS |
| DH82 cells + TNF siRNA | NO LPS BURSTS |
| DH82 cells + IL and TNF siRNA | NO LPS BURSTS |
| DH82 cells + negative control | NO LPS BURSTS |
| DH82 cells alone | NO LPS BURSTS |

Experiment 2: 4 Week Experiment (28 Days) with 2×24 hr LPS Bursts Per Week.

Experiment 1 was repeated and extended to 4 weeks. Media was collected after each LPS burst and at the completion of the experiment. For control cells without LPS exposure, media was collected and replaced with standard media at the same time points. At day 18 (week 3, immediately after LPS burst 5), the cells were passaged into larger T175 flasks (20 mls media). This was essential to maintain the general health of the cells by preventing overcrowding. After day 28 the media was collected and the cells were preserved for RNA isolation in Tri Reagent as before.

Experiment 3: 4 Week Experiment (28 Days) with or without Continual LPS Stimulation.

Experiment 2 was repeated but the 24 hour LPS challenges were replaced by continual stimulation with 1 μg/ml LPS. Media was collected and replaced once a week and at the completion of the experiment. After 28 days the cells were preserved for RNA isolation in Tri Reagent. Control cells without LPS stimulation were provided from Experiment 2.

RNA Isolation and Gene Expression Analysis

Total RNA was prepared from the harvested cells using Tri Reagent (Sigma) and RNeasy minicolumns and reagents (Qiagen Ltd., Surrey, UK) according to manufacturer's guidelines. An on-column DNase digestion step was included to ensure removal of residual genomic DNA. The RNA was quantified and complementary DNA (cDNA) was produced using 1 μg of total RNA per sample, via a standard reverse transcription (RT) reaction using random hexamer primers and the MMLV (Moloney murine leukaemia virus) RT enzyme (both Promega, Southampton, UK). Quantitative real time PCR (qRT-PCR), using an ABI 7300 real time PCR system (Warrington, UK) with Go Taq qRT-PCR Master Mix (Promega, Southampton, UK), was used to measure the mRNA levels of the target genes ADAMTS4, -5, COX2, IL-1β, IL-6, MMP13 and TNF. Canine specific GAPDH was used as the endogenous reference gene. Sequences of all primers are shown in Table 2.

IL-1β and TNF ELISAs

The release of IL-1β and TNF into the culture media was measured using commercially available canine specific ELISA kits according to the manufacturer's guidelines (2B Scientific and R and D Systems respectively). For experiment 1, the end media was assayed. For experiment 2, media collected after LPS challenges 1, 3, 5 and 7 (i.e. 4 weekly time points) and the end media was analysed. For experiment 3, the media collected at the weekly media change and the end media was analysed.

Results

In Vitro Bioactivity of siRNA-Loaded PLGA Microspheres

In the study, we designed three experiments with different LPS challenges to investigate the in vitro effects of siRNA-loaded PLGA microspheres.

Experiment 1: 8 Day Experiment with 2×24 hr LPS Bursts.

Gene Expression

FIG. 8-10 show the results the expression of key DH82 target genes, including ADAMTS4, ADAMTS5, COX2, IL-1 β, IL-6, MMP-13 and TNF (No stats have been done on the data.) The results show that the IL siRNA does not appear to have reduced IL-1β gene expression compared to the controls where the negative siRNA had been used or where no microspheres had been added (FIG. 10). The TNF siRNA may have slightly reduced TNF gene expression compared to the negative siRNA control.

IL-1β and TNF ELISA

FIG. 13-16 show the results of IL-1β and TNF protein released into the culture media from DH82 cells when cultured with or without two 24 hour LPS challenges at day 2 and day 6 for duration of eight days. The IL-1β siRNA does not appear to have reduced IL-1β release compared to cells cultured with the negative siRNA control or cells cultured with no microspheres. However, both siRNA loaded PLGA microspheres do have reduced TNF release compared to cells cultured with the negative siRNA or cells cultured with no microspheres under LPS challenges (FIG. 15).

Experiment 2: 28 Day Experiment with Two 24 Hour LPS Challenges, Twice Weekly. Gene Expression.

FIG. 17-19 show the expression of key DH82 target genes when cultured with and without LPS challenges (two per week) for duration of 28 days. The IL+TNF siRNA may have reduced IL and TNF expression compared to the controls (FIG. 17). However, when used individually the siRNAs have not reduced IL or TNF gene expression. The data suggest that expression was possibly reduced when no LPS was used (FIG. 17).

IL-1β and TNF ELISA

FIGS. 20-23 show the results of IL-1β and TNF protein released into the culture media from DH82 cells when cultured with or without two 24 hour LPS challenges per week for duration of 28 days. Compared to cells cultured without microspheres, the IL-1β appears to have been reduced (FIG. 20) with siRNA load PLGA microspheres after from 2 weeks to the end of the experiment with LPS challenges. TNF also appears to have been reduced at the end of the experiment with LPS challenges (FIG. 22). This is only evident when comparing to the control where no microspheres had been added and not when the negative control siRNA loaded microspheres were used suggesting that the microspheres themselves have an inflammatory effect.

Experiment 3: 28 Day Experiment with Continual LPS Stimulation.

Gene Expression.

FIGS. 24-26 show the expression of key DH82 target genes when cultured with and without continual LPS stimulation for duration of 28 days. The IL and TNF siRNAs have not reduced IL or TNF gene expression compared to the controls when the cells were subjected to continual LPS stimulation (FIG. 24). The results suggested that expression was reduced when no LPS was used (FIG. 25).

IL-1β and TNF ELISA

FIGS. 27-31 show the results of IL-1β and TNF protein released into the culture media from DH82 cells when cultured with or without continual LPS stimulation for duration of 28 days. The cumulative IL release chart (FIG. 29) demonstrates that IL-1β is reduced compared to cells cultured with no microspheres. However, the negative siRNA control has also reduced IL-1β release. By examining the weekly release, week 2 produces the largest difference between the IL siRNA and the cells alone. SiRNA loaded microspheres increased TNF production sharply in the week 1 (FIG. 30, 31).

### Discussion and Conclusions

An siRNA microsphere delivery system has been developed for intra-articular administration with sustained release of siRNA; targeting synovial macrophages with the clinical objective to treat osteoarthritis. SiRNA loaded PLGA microspheres have been successfully fabricated using an water-in-oil-in-water (W1/O/W2) solvent-evaporation technique with sonication. We have demonstrated the controlled-release of siRNA from microspheres for up to 6 weeks; with up to 63% siRNA being released in first 6 weeks. To adjust the siRNA release to be within 3 to 6 weeks, the PLGA blend ration can be adjusted to 50:50 or more complex multi ratio blend can be fabricated with PLGA 65:35 to finely tune the release rate and profile.

The efficiency and duration of TNF and IL-1β knockdown was also determined for the siRNA loaded small PLGA microspheres. This was evaluated by the measurement of the change in expression of key target genes and by measuring the amounts of TNF and IL-1β released in to media by DH82 cells with or without LPS challenge. It has been demonstrated that siRNA loaded microspheres decreased the release of IL-1β and TNF in DH82 challenged with LPS at day 8 and 2 weeks. However, there was no distinct difference in expression of key target genes and IL-1β/TNF release at other time points. In vivo experiments are now required to be conducted to evaluate the efficiency and longevity of siRNA loaded microspheres on TNF and IL-1β knockdown and the effects on osteoarthritis.

### Study 4

### In Vivo Effectiveness of the siRNAs of the Invention

The following study will be undertaken by the inventors to demonstrate the effectiveness of the siRNA molecules of the invention (provided in a method of the invention) in a canine model of synovitis. An LPS-induced model of synovitis will be used, in accordance with the literature in this field (see for example, Ross, et al. (2012) “Evaluation of the inflammatory response in experimentally induced synovitis in the horse: a comparison of recombinant equine interleukin 1β and lipopolysaccharide.” Osteoarthritis and Cartilage 20, 1583-1590).

Experimental Design

Animals:

The study will be undertaken using 10 healthy adult dogs (preferably Labrador type dogs; 5 experimental, 5 controls) that are acclimated to walking across a force platform and are known to have symmetric gait cycles.

Experimental Therapeutic Agents:

The study will utilise siRNAs of the invention formulated in PLGA microspheres, referred to as “siRNA ms” in the summary Table 3 below.

Primary Outcomes Measure:

Peak vertical force, as measured in the trotting dog (2.2-2.4 m/s) on a force platform and expressed as N/Kg bodyweight.

Secondary Outcomes Measures:

The following will be assessed, and any of these may be included as measurements of secondary outcomes
Synovial fluid TNF protein as measured by ELISA (R&D Systems)
Synovial fluid IL-1B protein as measured by ELISA (Kingfisher Biotech)
Synovial fluid COX-2 protein as measured by ELISA
Synovial fluid cell pellet gene expression for TNF and IL-1B as measured by qRT-PCR
Synovial histology
Synovial gene expressions analysis

A summary of this study is set out in Table 3.

TABLE 1
Primers for quantitative RT-PCR for canine genes
investigated in Study 1

| Gene | Primer (F-forward; R-reverse) |
|---|---|
| ADAMTS-5 | F - TTA CGA GAG AGG ATT TAT GTG GGC |
| | (SEQ ID NO. 13) |
| | R - CGC TTA TCT TCT GTG GAA CCA AAG |
| | (SEQ ID NO. 14) |
| ADAMTS-4 | F - TGCTGTTGTGGAGGATGATGG |
| | (SEQ ID NO. 15) |
| | R - GAACTCAGTGATGAAGTGGGCAC |
| | (SEQ ID NO. 16) |
| GAPDH | F - GTC CAC TGG TGT CTT CAC TAC CTT G |
| | (SEQ ID NO. 17) |
| | R - CAC AAA CAT TGG GGC ATC AGC |
| | (SEQ ID NO. 18) |
| IL-1β | F - ATGTGAAGTGCTGCTGCCAAG |
| | (SEQ ID NO. 19) |
| | R - CAATGACTGACACGAAATGCCTC |
| | (SEQ ID NO. 20) |
| MMP-13 | F - AAC TTG TTC CTT GTC GCT GCC |
| | (SEQ ID NO. 21) |
| | R - GTT TTG GGA TGT CTG GGG TTG |
| | (SEQ ID NO. 22) |
| PTGS2 | F - TTC AAG GGA GTC TGG AAC ATT GTC |
| | (SEQ ID NO. 23) |
| | R - TCA TCA GGC ACA GGA GGA AGA G |
| | (SEQ ID NO. 24) |
| TNF | F - GCC TAA CTA TCT GGA CTT TGC CG |
| | (SEQ ID NO. 25) |
| | R - TTT CTA AGC CTG AAG GGG GTG AGG |
| | (SEQ ID NO. 26) |

TABLE 2
Primers for quantitative RT-PCR for canine genes investigated
in Study 2

| Gene | Forward primer | Reverse primer |
|---|---|---|
| ADAMTS4 | TGCTGTTGTGGAGGATGATGG | GAACTCAGTGATGAAGTGGGCAC |
| | (SEQ ID NO. 27) | (SEQ ID NO. 28) |
| ADAMTS5 | TTACGAGAGAGGATTTATGTGGGC | TTACGAGAGAGGATTTATGTG GGC |
| | (SEQ ID NO. 29) | (SEQ ID NO. 30) |
| COX2 | TTCAAGGGAGTCTGGAACATTGTC | TCATCAGGCACAGGAGGAAGAG |
| | (SEQ ID NO. 31) | (SEQ ID NO. 32) |
| IL-1β | ATGTGAAGTGCTGCTGCCAAG | CAATGACTGACACGAAATGCCTC |
| | (SEQ ID NO. 33) | (SEQ ID NO. 34) |
| IL-6 | GGCTACTGCTTTCCCTACCC | TTTTCTGCCAGTGCCTCTTT |
| | (SEQ ID NO. 35) | (SEQ ID NO. 36) |
| MMP13 | AACTTGTTCCTTGTCGCTGCC | GTTTTGGGATGTCTGGGGTTG |
| | (SEQ ID NO. 37) | (SEQ ID NO. 38) |
| TNF | GGTCAACCTACTCTCTCTGCCATCAAG | TTTCTAAGCCTGAAGGGGGTGAGG |
| | (SEQ ID NO. 39) | (SEQ ID NO. 40) |
| GAPDH | GTCCACTGGTGTCTTCACTACCTTG | CACAAACATTGGGGCATCAGC |
| | (SEQ ID NO. 41) | (SEQ ID NO. 42) |

TABLE 3
Experimental plan summary for Study 4

| | Intervention | | |
|---|---|---|---|
| Day | Experimental treated | Control | Measurement |
| 1 | Aspirate synovial fluid | Aspirate SF | Clinical examination |
| | (SF) | Inject 0.25 ng 0.5 ng LPS | Baseline force platform |
| | Inject 0.25 ng 0.5 ng | (from E. coli 055:B5; | measures |
| | LPS (from E. coli | Sigma Aldrich) only in to | Repeat force platform (FP) |
| | 055:B5; Sigma Aldrich) | right stifle joint | measures at 8 h post-injection |
| | AND siRNA ms in to | | |
| | right stifle joint | | |
| 2 | | | Repeat FP measures |
| 3 | Aspirate SF | Aspirate SF | Repeat FP measures |
| 14 | Aspirate SF | Aspirate SF | Clinical examination |
| | Inject 0.25 ng 0.5 ng | Inject 0.25 ng 0.5 ng LPS | Repeat force platform |
| | LPS (from E. coli | (from E. coli 055:B5; | measures |
| | 055:B5; Sigma Aldrich) | Sigma Aldrich) only in to | |
| | only in to right stifle | right stifle joint | |
| | joint | | |
| 15 | | | Repeat FP measures |
| 16 | Aspirate SF | Aspirate SF | Repeat FP measures |
| 28 | Aspirate SF | Aspirate SF | Clinical examination |
| | Inject 0.25 ng 0.5 ng | Inject 0.25ng 0.5 ng LPS | Repeat force platform |
| | LPS (from E. coli | (from E. coli 055:B5; | measures |
| | 055:B5; Sigma Aldrich) | Sigma Aldrich) only in to | |
| | only in to right stifle | right stifle joint | |
| | joint | | |
| 29 | | | Repeat FP measures |
| 30 | Aspirate SF | Aspirate SF | Repeat FP measures |
| 42 | Aspirate SF | Aspirate SF | Clinical examination |
| | Inject 0.25 ng 0.5 ng | Inject 0.25 ng 0.5 ng LPS | Repeat force platform |
| | LPS (from E. coli | (from E. coli 055:B5; | measures |
| | 055:B5; Sigma Aldrich) | Sigma Aldrich) only in to | |
| | only in to right stifle | right stifle joint | |
| | joint | | |
| 43 | | | Repeat FP measures |
| 44 | Aspirate SF | Aspirate SF | Repeat FP measures |
| | Euthanasia | Euthanasia | Post mortem |
| | | | Collect and store tissues for |
| | | | histology and gene |
| | | | expression |

Improvements in the primary outcomes measure, and optionally in one or more of the secondary outcomes measures, will provide further illustration of the therapeutic effectiveness of the siRNAs of the invention in vivo.

TABLE 4
Exemplary TNF silencing siRNA molecules of the
invention, as used in knock-down experiments
described herein

| Target | | |
|---|---|---|
| gene | siRNA molecule | siRNA sequences |
| TNF | siRNA-TNF-A | GGCUUAGAAAGAGAAUUAAtt |
| | | SEQ ID NO. 4 |
| | | UUAAUUCUCUUUCUAAGCCtg |
| | | SEQ ID NO. 1 |
| TNF | siRNA-TNF-B | GGUGUACUUUGGAAUCAUUtt |
| | | SEQ ID NO. 5 |
| | | AAUGAUUCCAAAGUACACCtg |
| | | SEQ ID NO. 2 |
| TNF | siRNA-TNF-C | GCCUAACUAUCUGGACUUUtt |
| | | SEQ ID NO. 6 |
| | | AAAGUCCAGAUAGUUAGGCag |
| | | SEQ ID NO. 3 |

TABLE 5
Exemplary IL-1β silencing siRNA molecules
of the invention, as used in knock-down
experiments described herein

| Target | | |
|---|---|---|
| gene | siRNA molecule | siRNA sequences |
| IL-1β | siRNA-1L1β-A | GCUACAUCUUUGAAGAAGAtt |
| | | SEQ ID NO. 10 |
| | | UCUUCUUCAAAGAUGUAGCaa |
| | | SEQ ID NO. 7 |
| IL-1β | siRNA-IL1β-B | CGAUUUGUCUUCAACAAGAtt |
| | | SEQ ID NO. 11 |
| | | UCUUGUUGAAGACAAAUCGct |
| | | SEQ ID NO. 8 |
| IL-1β | siRNA-IL1β-C | CACCAGCUCUGUAACAAGAtt |
| | | SEQ ID NO. 12 |
| | | UCUUGUUACAGAGCUGGUGgg |
| | | SEQ ID NO. 9 |

The siRNA molecules set out in Table 4 may be used in combination with the siRNA molecules of the invention set out in Table 5 in any aspects or embodiments of the invention.

1. An siRNA molecule comprising a nucleic acid sequence selected from the group consisting of: SEQ ID NO. 1, SEQ ID NO. 2, SEQ ID NO. 3, SEQ ID NO. 7, SEQ ID NO. 8, SEQ ID NO. 9 or a nucleic acid sequence sharing at least 90% identity with SEQ ID NO. 1, SEQ ID NO. 2, SEQ ID NO. 3, SEQ ID NO. 7, SEQ ID NO. 8, SEQ ID NO. 9.

2. An siRNA molecule according to claim 1 consisting of a nucleic acid sequence selected from the group consisting of: SEQ ID NO. 1, SEQ ID NO. 2, SEQ ID NO. 3, SEQ ID NO. 7, SEQ ID NO. 8, SEQ ID NO. 9 or a nucleic acid sequence sharing 90% identity with SEQ ID NO. 1, SEQ ID NO. 2, SEQ ID NO. 3, SEQ ID NO. 7, SEQ ID NO. 8, SEQ ID NO. 9

3. An siRNA molecule according to claim 1, that differs from SEQ ID NO. 1, SEQ ID NO. 2, SEQ ID NO. 3, SEQ ID NO. 7, SEQ ID NO. 8 or SEQ ID NO. 9 by a maximum of two nucleotides.

4. An siRNA molecule according to claim 3 that differs from SEQ ID NO. 1, SEQ ID NO. 2, SEQ ID NO. 3, SEQ ID NO. 7, SEQ ID NO. 8 or SEQ ID NO. 9 by a single nucleotide.

5. An siRNA molecule according to claim 1, comprising a nucleic acid sequence selected from a group consisting of SEQ ID NO. 2 or SEQ ID NO. 7 or a nucleic acid sequence sharing 90% identity with SEQ ID NO. 2 or SEQ ID NO. 7.

6. An siRNA molecule according to claim 1, wherein the siRNA molecule is a tumour necrosis factor-silencing siRNA molecule, and is selected from the group of nucleic acid molecules comprising SEQ ID NO. 1, SEQ ID NO. 2, SEQ ID NO. 3 and nucleic acid molecules based on these sequences.

7. An siRNA molecule according to claim 1, wherein the siRNA molecule is an interleukin 1-β-silencing siRNA molecule, and is selected from the group of nucleic acid molecules comprising SEQ ID NO. 7, SEQ ID NO. 8, SEQ ID NO. 9 and nucleic acid molecules based on these sequences.

8-16. (canceled)

17. A pharmaceutical composition comprising an siRNA molecule according to claim 1 and a pharmaceutically acceptable carrier.

18. A pharmaceutical composition according to claim 17, wherein the pharmaceutically acceptable carrier comprises PLGA microspheres.

19. A pharmaceutical composition according to claim 17, wherein the PLGA microspheres encapsulate the siRNA molecules.

20. A pharmaceutical composition according to claim 17, wherein the PLGA microspheres have a diameter of between about 1 μm and 60 μm.

21. A pharmaceutical composition according to claim 20, wherein the PLGA microspheres have a diameter of between about 2 μm and 50 μm.

22. A pharmaceutical composition according to claim 21, wherein the PLGA microspheres have a diameter of between about 2 μm and 10 μm.

23. A pharmaceutical composition according to claim 22, wherein the PLGA microspheres have a diameter of between about 2 μm and 5 μm.

24. A pharmaceutical composition according to claim 17, further comprising an additional therapeutic agent.

25. A pharmaceutical composition according to claim 24, wherein the additional therapeutic agent comprises a further siRNA molecule.

26. A pharmaceutical composition according to claim 25, comprising an siRNA molecule selected from the group of nucleic acid molecules comprising SEQ ID NO. 1, SEQ ID NO. 2, SEQ ID NO. 3 and nucleic acid molecules based on these sequences, and an siRNA molecule selected from the group of nucleic acid molecules comprising SEQ ID NO. 7, SEQ ID NO. 8, SEQ ID NO. 9 and nucleic acid molecules based on these sequences.

27. (canceled)

28. A method of preventing and/or treating osteoarthritis in a dog, the method comprising providing to a dog in need of such prevention and/or treatment a therapeutically effective amount of an siRNA molecule according to claim 1.

29. A method according to claim 28, wherein the siRNA molecule is provided in the form of a pharmaceutical composition comprising an siRNA molecule comprising a nucleic acid sequence selected from the group consisting of: SEQ ID NO. 1, SEQ ID NO. 2, SEQ ID NO. 3, SEQ ID NO. 7, SEQ ID NO. 8, SEQ ID NO. 9 or a nucleic acid sequence sharing at least 90% identity with SEQ ID NO. 1, SEQ ID NO. 2, SEQ ID NO. 3, SEQ ID NO. 7, SEQ ID NO. 8, SEQ ID NO. 9, and a pharmaceutically acceptable carrier.

30. A method according to claim 28, wherein providing to the dog is via localised delivery to a site requiring prevention and/or treatment of osteoarthritis.

31. A method according to claim 28, wherein providing to the dog is via injection into a joint requiring prevention and/or treatment of osteoarthritis.

32. A method according to claim 28, wherein the siRNA molecule is provided in a combination therapy to the dog for prevention and/or treatment of osteoarthritis.

33. A method according to claim 28, wherein the siRNA molecule is selected from the group of nucleic acid molecules comprising SEQ ID NO. 1, SEQ ID NO. 2, SEQ ID NO. 3 and nucleic acid molecules based on these sequences, and used in combination with an siRNA molecule selected from the group of nucleic acid molecules comprising SEQ ID NO. 7, SEQ ID NO. 8, SEQ ID NO. 9 and nucleic acid molecules based on these sequences.

34. A method according to claim 28, wherein the siRNA molecule is selected from the group of nucleic acid molecules comprising SEQ ID NO. 7, SEQ ID NO. 8, SEQ ID NO. 9 and nucleic acid molecules based on these sequences, and is used in combination with an siRNA molecule selected from the group of nucleic acid molecules comprising SEQ ID NO. 1, SEQ ID NO. 2, SEQ ID NO. 3 and nucleic acid molecules based on these sequences.

35. A method according to claim 28, wherein the siRNA molecule is used in the reduction of the likelihood of osteoarthritis, and is provided to the dog prior to the onset of osteoarthritis.

36. A method according to claim 28, wherein the siRNA molecule is used in the treatment of osteoarthritis, and is provided to a dog diagnosed as having osteoarthritis.

37. A method according to claim 28, wherein a combination of siRNA molecules comprising SEQ ID NO.2, or nucleic acid molecules based on this sequence, and siRNA molecules comprising SEQ ID NO.7, or nucleic acid molecules based on this sequence is provided to the dog.

38. A method of preventing and/or treating osteoarthritis in a dog, the method comprising providing to a dog in need of such prevention and/or treatment a therapeutically effective amount of a pharmaceutical composition according to claim 26, comprising a combination of siRNA molecules comprising SEQ ID NO.2, or nucleic acid molecules based on this sequence, and siRNA molecules comprising SEQ ID NO.7, or nucleic acid molecules based on this sequence.


SEQUENCE LISTING (42 sequences)

SEQ ID NO: 1 - DNA, 21 bp, Artificial Sequence
  siRNA
uuaauucucuuucuaagcctg

SEQ ID NO: 2 - DNA, 21 bp, Artificial Sequence
  siRNA
aaugauuccaaaguacacctg

SEQ ID NO: 3 - DNA, 21 bp, Artificial Sequence
  siRNA
aaaguccagauaguuaggcag

SEQ ID NO: 4 - DNA, 21 bp, Artificial Sequence
  siRNA
ggcuuagaaagagaauuaatt

SEQ ID NO: 5 - DNA, 21 bp, Artificial Sequence
  siRNA
gguguacuuuggaaucauutt

SEQ ID NO: 6 - DNA, 21 bp, Artificial Sequence
  siRNA
gccuaacuaucuggacuuutt

SEQ ID NO: 7 - DNA, 21 bp, Artificial Sequence
  siRNA
ucuucuucaaagauguagcaa

SEQ ID NO: 8 - DNA, 21 bp, Artificial Sequence
  siRNA
ucuuguugaagacaaaucgct

SEQ ID NO: 9 - DNA, 21 bp, Artificial Sequence
  siRNA
ucuuguuacagagcugguggg

SEQ ID NO: 10 - DNA, 21 bp, Artificial Sequence
  siRNA
gcuacaucuuugaagaagatt

SEQ ID NO: 11 - DNA, 21 bp, Artificial Sequence
  siRNA
cgauuugucuucaacaagatt

SEQ ID NO: 12 - DNA, 21 bp, Artificial Sequence
  siRNA
caccagcucuguaacaagatt

SEQ ID NO: 13 - DNA, 24 bp, Artificial Sequence
  RT-PCT Primer Sequence
ttacgagagaggatttatgtgggc

SEQ ID NO: 14 - DNA, 24 bp, Artificial Sequence
  RT-PCT Primer Sequence
cgcttatcttctgtggaaccaaag

SEQ ID NO: 15 - DNA, 21 bp, Artificial Sequence
  RT-PCT Primer Sequence
tgctgttgtggaggatgatgg

SEQ ID NO: 16 - DNA, 23 bp, Artificial Sequence
  RT-PCT Primer Sequence
gaactcagtgatgaagtgggcac

SEQ ID NO: 17 - DNA, 25 bp, Artificial Sequence
  RT-PCT Primer Sequence
gtccactggtgtcttcactaccttg

SEQ ID NO: 18 - DNA, 21 bp, Artificial Sequence
  RT-PCT Primer Sequence
cacaaacattggggcatcagc

SEQ ID NO: 19 - DNA, 21 bp, Artificial Sequence
  RT-PCT Primer Sequence
atgtgaagtgctgctgccaag

SEQ ID NO: 20 - DNA, 23 bp, Artificial Sequence
  RT-PCT Primer Sequence
caatgactgacacgaaatgcctc

SEQ ID NO: 21 - DNA, 21 bp, Artificial Sequence
  RT-PCT Primer Sequence
aacttgttccttgtcgctgcc

SEQ ID NO: 22 - DNA, 21 bp, Artificial Sequence
  RT-PCT Primer Sequence
gttttgggatgtctggggttg

SEQ ID NO: 23 - DNA, 24 bp, Artificial Sequence
  RT-PCT Primer Sequence
ttcaagggagtctggaacattgtc

SEQ ID NO: 24 - DNA, 22 bp, Artificial Sequence
  RT-PCT Primer Sequence
tcatcaggcacaggaggaagag

SEQ ID NO: 25 - DNA, 23 bp, Artificial Sequence
  RT-PCT Primer Sequence
gcctaactatctggactttgccg

SEQ ID NO: 26 - DNA, 24 bp, Artificial Sequence
  RT-PCT Primer Sequence
tttctaagcctgaagggggtgagg

SEQ ID NO: 27 - DNA, 21 bp, Artificial Sequence
  RT-PCT Primer Sequence
tgctgttgtggaggatgatgg

SEQ ID NO: 28 - DNA, 23 bp, Artificial Sequence
  RT-PCT Primer Sequence
gaactcagtgatgaagtgggcac

SEQ ID NO: 29 - DNA, 24 bp, Artificial Sequence
  RT-PCT Primer Sequence
ttacgagagaggatttatgtgggc

SEQ ID NO: 30 - DNA, 24 bp, Artificial Sequence
  RT-PCT Primer Sequence
ttacgagagaggatttatgtgggc

SEQ ID NO: 31 - DNA, 24 bp, Artificial Sequence
  RT-PCT Primer Sequence
ttcaagggagtctggaacattgtc

SEQ ID NO: 32 - DNA, 22 bp, Artificial Sequence
  RT-PCT Primer Sequence
tcatcaggcacaggaggaagag

SEQ ID NO: 33 - DNA, 21 bp, Artificial Sequence
  RT-PCT Primer Sequence
atgtgaagtgctgctgccaag

SEQ ID NO: 34 - DNA, 23 bp, Artificial Sequence
  RT-PCT Primer Sequence
caatgactgacacgaaatgcctc

SEQ ID NO: 35 - DNA, 20 bp, Artificial Sequence
  RT-PCT Primer Sequence
ggctactgctttccctaccc

SEQ ID NO: 36 - DNA, 20 bp, Artificial Sequence
  RT-PCT Primer Sequence
ttttctgccagtgcctcttt

SEQ ID NO: 37 - DNA, 21 bp, Artificial Sequence
  RT-PCT Primer Sequence
aacttgttccttgtcgctgcc

SEQ ID NO: 38 - DNA, 21 bp, Artificial Sequence
  RT-PCT Primer Sequence
gttttgggatgtctggggttg

SEQ ID NO: 39 - DNA, 27 bp, Artificial Sequence
  RT-PCT Primer Sequence
ggtcaacctactctctctgccatcaag

SEQ ID NO: 40 - DNA, 24 bp, Artificial Sequence
  RT-PCT Primer Sequence
tttctaagcctgaagggggtgagg

SEQ ID NO: 41 - DNA, 25 bp, Artificial Sequence
  RT-PCT Primer Sequence
gtccactggtgtcttcactaccttg

SEQ ID NO: 42 - DNA, 21 bp, Artificial Sequence
  RT-PCT Primer Sequence
cacaaacattggggcatcagc